(19) United States
(12) Patent Application Publication
(10) Pub. No.: US 2025/0160730 A1
(43) Pub. Date: May 22, 2025

(54) Portable Radio Frequency Medical Imaging Devices, Systems, and Methods

(71) Applicant: Clean Imager Inc., Fremont, CA (US)

(72) Inventor: Stephen Pearce, Fremont, CA (US)

(21) Appl. No.: 18/955,098

(22) Filed: Nov. 21, 2024

Related U.S. Application Data
(60) Provisional application No. 63/601,372, filed on Nov. 21, 2023.

(51) Int. Cl. A61B 5/00; G06T 7/00; G06T 7/10

(52) U.S. Cl. CPC A61B 5/4088; G06T 7/0016; G06T 7/10; G06T 2207/30016

(57) ABSTRACT

A portable medical imaging device includes a housing defining an interior cavity sized to receive a head of a patient. The device also includes a radio frequency absorbing material positioned in the interior cavity of the housing and a matching liquid configured to be positioned between the radio frequency absorbing material and the patient's head received within the cavity of the housing. The device also includes a radio frequency array including a plurality of ultra-wide band (UWB) transceivers distributed in the radio frequency absorbing material configured to direct radio frequency signals towards the patient's head and to detect radio frequency signals emitted by other transceivers of the plurality of UWB transceivers; and at least one controller. The at least one controller is configured to receive and process information about the radio frequency signals detected by the plurality of UWB transceivers for generation of medical images of the patient's head.

### CROSS REFERENCE TO RELATED APPLICATION

The present application claims the benefit of U.S. Provisional Patent Application No. 63/601,372 filed Nov. 21, 2023, the disclosure of which is hereby incorporated by reference in its entirety.

### BACKGROUND OF THE INVENTION

### Field of the Invention

The present disclosure is directed to medical imaging devices, systems, and methods for obtaining images of internal body structures including the brain, internal organs, limbs, soft tissues and bone, as well as for imaging of internal injuries, diseased tissues (e.g. Alzheimer's affected tissues), tumors, wounds, and similar conditions, and, in particular, to a portable radio frequency imaging device for neuro-imaging applications, as well as to portable radio frequency imaging devices for imaging of the torso, abdomen, arms, legs, and/or for whole body imaging.

### Description of Related Art

Medical images of brain tissue can be analyzed for a variety of diagnostic purposes including for Alzheimer's screening and amyloid rated imaging abnormalities (ARIA) monitoring, as well as for evaluation of traumatic brain injury (TBI), tumor screening, and/or for detection of stroke, migraines, concussions, and similar injuries and conditions. Computed Tomography (CT) and Magnetic Resonance Imaging (MRI) are common medical imaging techniques used for detection and diagnosis of brain injuries and conditions, as well as for detection and monitoring of other conditions throughout the body. Of particular benefit, CT/MRI can be used for obtaining three-dimensional (3D) slice-based images with millimetric resolution. However, accessibility of CT and MRI imaging can be limited to large medical facilities and/or neurological treatment facilities, meaning that imaging may not be available in many situations where CT/MRI images would be useful for guiding patient therapy. Also, such imaging generally is not available in emergency situations or outside of a medical facility.

Recently, it has been recognized that brain imaging can be used for targeting drug therapy for Alzheimer's treatment (e.g., drug therapy with lecanemab targeting amyloid beta plaques) leading to improved outcomes with regards to reduced cognitive and functional decline for patients. However, treatment with lecanemab may require monitoring for ARIA (Amyloid-Related Imaging Abnormalities), such as clusters of small hemorrhages, which are a side effect of this drug therapy. Consistent brain imaging can be used to determine when dosage changes are needed to prevent brain injury. Often such imaging involves frequent MRI brain scans. See Expert discusses recent lecanemab trial, why it appears to offer hope for those with deadly disease, The Harvard Gazette, http://news.harvard.edu/gazette/story/2023/06/start-of-new-era-for-alzheimers-treatment/(June 2023). This need for frequent MRI scans can be an impediment limiting the number of patients who can be treated using this drug therapy due to high cost and limited accessibility of current MRI scanning equipment. For example, it has been estimated that there may be 7 million new patients suffering from Alzheimer's who would benefit from drug therapy with lecanemab. Each of the 7 million patients may require 5-12 MRI scans annually according to lecanemab prescribed standard-of-care requirements. Thus, the 7 million new patients would require approximately 35-84 million new MRI scans per year. This number far exceeds the current capacity of 40 million MRI scans performed in the United States annually for all indications. As such, accessibility of MRI scans may be a significant barrier to widespread adoption of drug therapy for Alzheimer's treatment.

In addition to limited capacity for such scans, damaging effects of CT/MRI are well known. For example, CT uses ionizing radiation, which can present a danger to patients and practitioners. MRI uses high power radio frequency (RF) and high level magnetic fields, which may not be compatible with metal implants and medical devices comprising metals. Furthermore, for some imaging procedures, CT and MRI require injection of contrast agents, which can cause undesirable reactions. Also, CT and MRI can be expensive and may only be performed at medical facilities with CT and MRI imaging devices, which limits how frequently images can be acquired for some patients. In particular, CT/MRI capabilities are not ubiquitous in emergency departments and are rarely used during emergency rescue situations. In addition, MRI suffers from a long examination time. CT can be gathered more quickly (e.g., minutes). However, often a patient must wait hours before CT imaging can be scheduled and performed. Furthermore, ischemia can be difficult to interpret from CT images alone, making it difficult to determine an actionable clinical plan based solely on CT images and without waiting for some tissue (e.g., brain) damage to become visible.

In view of such difficulties with CT/MRI imaging, the imaging devices and methods disclosed herein are intended to provide a safe and highly portable alternative to CT/MRI for use in obtaining images for diagnosis upon which treatment can be based.

### SUMMARY OF THE INVENTION

According to an aspect of the disclosure, a portable medical imaging device includes a housing defining an interior cavity sized to receive a head of a patient. The device also includes a radio frequency absorbing material positioned in the interior cavity of the housing and a radio frequency matching liquid configured to be positioned between the radio frequency absorbing material and the patient's head received within the cavity of the housing. The device also includes a radio frequency array including a plurality of ultra-wide band (UWB) transceivers distributed in the radio frequency absorbing material configured to direct radio frequency signals towards the patient's head and to detect radio frequency signals emitted by other transceivers of the plurality of UWB transceivers and at least one controller. The at least one controller is configured to receive and process information about the radio frequency signals detected by the plurality of UWB transceivers for generation of medical images of the patient's head.

According to another aspect of the disclosure, a medical imaging system includes at least one portable medical imaging device including any features described herein and a wireless transmitter in communication with at least one controller of the at least one portable medical imaging device. The system also includes at least one remote computing device configured to wirelessly receive data from the at least one portable medical imaging device and to generate one or more medical images based on measurement data wirelessly received from the at least one portable medical imaging device.

According to another aspect of the disclosure, a portable medical imaging device includes a blanket with an outwardly facing surface and an inwardly facing surface configured to be positioned proximate to a patient; a radio frequency absorbing material positioned in the blanket between the inwardly facing surface and the outwardly facing surface of the blanket; and a radio frequency matching liquid configured to be positioned between the radio frequency absorbing material and the patient, while the patient is covered by the blanket. The device also includes a radio frequency array comprising a plurality of ultra-wide band (UWB) transceivers distributed in the radio frequency absorbing material configured to direct radio frequency signals to the patient and to detect radio frequency signals emitted by other transceivers of the plurality of UWB transceivers and at least one controller. The at least one controller is configured to receive and process information about signals detected by the plurality of UWB transceivers to generate medical images.

According to another aspect of the disclosure, a tracking and data collection system for medical imaging includes a plurality of portable medical imaging devices including any of the features described herein and a computer server. The computer server is configured to receive and process signals communicated from the plurality of imaging devices, determine a location for one or more of the plurality of portable medical imaging devices based on the received and processed signals, generate medical images based on the received and processed signals, and store generated images in electronic medical records associated with one or more of a plurality of patients.

According to another aspect of the disclosure, a method of triage for a patient who has suffered and/or is suffering a stroke includes steps of capturing at least one medical image of a head of the patient using a portable medical imaging device including any of the features described herein and analyzing the captured at least one medical image to distinguish between areas of the at least one captured image evidencing an ischemic stroke and a hemorrhagic stroke. The method also includes treating the patient for either the identified ischemic stroke or the identified hemorrhagic stroke.

According to another aspect of the disclosure, a method of treatment for a patient suspected of having and/or diagnosed with Alzheimer's disease includes steps of capturing at least one medical image of the patient using a portable medical imaging device including any of the features described herein and analyzing the captured at least one medical image to determine a characteristic of Alzheimer's tissue visible in the captured at least one medical image. The method also includes a step of providing at least one medication at a dosage determined based, at least in part, on the analysis of the captured image.

Non-limiting examples of the present invention will now be described in the following numbered clauses:

Clause 1: A portable medical imaging device, comprising: a housing defining an interior cavity sized to receive a head of a patient; a radio frequency absorbing material positioned in the interior cavity of the housing; a radio frequency matching liquid configured to be positioned between the radio frequency absorbing material and the patient's head received within the cavity of the housing; a radio frequency array comprising a plurality of ultra-wide band (UWB) transceivers distributed in the radio frequency absorbing material configured to direct radio frequency signals towards the patient's head and to detect the radio frequency signals emitted by other transceivers of the plurality of UWB transceivers; and at least one controller configured to receive and process information about the radio frequency signals detected by the plurality of UWB transceivers for generation of medical images of the patient's head.

Clause 2: The portable medical imaging device of clause 1, wherein the housing comprises a helmet configured to be used for at least one of the following diagnostic purposes: Alzheimer's screening, amyloid rated imaging abnormalities (ARIA) monitoring, evaluation of traumatic brain injury (TBI), tumor screening, detection of stroke, detection of migraines, or detection of concussions, the helmet comprising a shell configured to partially enclose the head of the patient.

Clause 3: The portable medical imaging device of clause 2, wherein the shell comprises a metal material.

Clause 4: The portable medical imaging device of clause 2 or clause 3, wherein the helmet comprises a front clam-shell portion configured to cover at least a forehead and crown of the head and a rear clam-shell portion pivotally connected to the front clam-shell portion configured to cover an occipital portion of the head.

Clause 5: The portable medical imaging device of any of clauses 1-4, wherein the helmet further comprises a neck extension extending downward from the shell configured to cover a neck, shoulders, and/or clavicle of the patient for imaging areas of the neck, shoulders, and/or clavicle area of the patient.

Clause 6: The portable medical imaging device of clause 2 or clause 3, wherein the helmet comprises a window configured to allow the patient to see objects and/or people outside of the helmet.

Clause 7: The portable medical imaging device of clause 6, wherein the window comprises an opening in a shell of the helmet covered by a transparent or translucent panel.

Clause 8: The portable medical imaging device of clause 7, wherein the panel comprises a material configured to block the radio frequency signals from the plurality of UWB transceivers.

Clause 9: The portable medical imaging device of clause 6 or clause 7, wherein none of the plurality of UWB transceivers cover the window.

Clause 10: The portable medical imaging device of clause 9, wherein the matching liquid covers the window, the matching liquid being clear or substantially clear allowing the patient to see through the matching liquid and the window.

Clause 11: The portable medical imaging device of any of clauses 1-10, further comprising a visor configured to be within the field of view of the patient when wearing the imaging device, the visor comprising a visual display screen configured to display entertainment content to the patient during an imaging procedure.

Clause 12: The portable medical imaging device of any of clauses 1-11, wherein the imaging device is configured to be carried by a single staff member of a medical facility, and/or wherein the imaging device weighs 50 pounds or less, 40 pounds or less, or preferably about 36 pounds or less.

Clause 13: The portable medical imaging device of any of clauses 1-12, further comprising one or more air channels extending through the housing to the interior allowing breathable air to pass through the housing to the patient for breathing.

Clause 14: The portable medical imaging device of any of clauses 1-13, wherein the radio frequency absorbing material comprises doped rubber supporting the plurality of UWB transceivers.

Clause 15: The portable medical imaging device of any of clauses 1-14, further comprising one or more polymeric membranes separating the absorbing liquid from the matching liquid.

Clause 16: The portable medical imaging device of any of clauses 1-15, wherein the matching liquid comprises a liquid material having a permittivity configured to match brain permittivity and positioned to avoid air gaps between the plurality of UWB transceivers and the patient.

Clause 17: The portable medical imaging device of any of clauses 1-16, further comprising a compressible celled support comprising a plurality of cells containing the matching liquid and a liquid reservoir in fluid communication with the plurality of cells configured so that matching liquid passes between the plurality of cells and the reservoir to accommodate heads of different sizes.

Clause 18: The portable medical imaging device of clause 17, wherein the compressible celled support comprises rubberized walls defining substantially cube-shaped cells.

Clause 19: The portable medical imaging device of clause 18, wherein the compressible celled structure comprises a honeycomb enclosure and wherein the reservoir comprises a donut shaped enclosure configured to be positioned around the patient's head and in fluid communication with the plurality of cells.

Clause 20: The portable medical imaging device of any of clauses 1-19, wherein the radio frequency array comprises at least 300 UWB transceivers.

Clause 21: The portable medical imaging device of any of clauses 1-20, wherein the radio frequency array produces an image based on at least 300 by 300 RF signal measurements.

Clause 22: The portable medical imaging device of any of clauses 1-21, wherein the plurality of UWB transceivers operate at a frequency of about 3 GHz to about 8 GHz, and/or wherein the plurality of UWB transceivers operate with a bandwidth of at least 500 MHz, and preferably with an operating center frequency within a range of about 3.5 GHz to 9 GHz.

Clause 23: The portable medical imaging device of any of clauses 1-22, wherein the at least one controller is configured to generate images having millimetric resolution and substantially or close to zero latency based on the received and processed information.

Clause 24: The portable medical imaging device of any of clauses 1-23, wherein the received and processed information comprises information representative of times-of-flight between pairs of the plurality of UWB transceivers, which are representative of materials through which propagation of the radio frequency signals has occurred.

Clause 25: The portable medical imaging device of clause 24, wherein the at least one controller is configured to generate one or more of the medical images based on comparisons between the times-of-flight between the pairs of the UWB transceivers and possible times of fight calculated by a simulated model of possible times-of-flight representative of relative permittivity of tissue.

Clause 26: The portable medical imaging device of any of clauses 1-25, wherein the at least one controller is configured to cause the plurality of UWB transceivers to emit pulses of radio frequency signals according to a predetermined impulse response pattern and to detect the radio frequency signals traveling along paths between predetermined pairs of the plurality of UWB transceivers.

Clause 27: The portable medical imaging device of clause 26, wherein the impulse response pattern comprises a predetermined impulse response pattern selected to permit multiple internal reflections, wherein some of the multiple internal reflections are damped by absorption reducing a number of paths between pairs of the plurality of USB transceivers required for the generation of the medical images.

Clause 28: The portable medical imaging device of any of clauses 1-27, wherein the at least one controller is configured to generate the medical images by applying a transfer function to the received and processed information about the radio frequency signals detected by the plurality of UWB transceivers, wherein the transfer function comprises a function determined by modeling images established for possible image cases detectable by the radio frequency array.

Clause 29: The portable medical imaging device of clause 28, wherein the transfer function is based on relationships between material properties of tissue within paths between pairs of the plurality of UWB transceivers identified in the modeled images and time/power measurements identified in information detected by the plurality of USB transceivers.

Clause 30: The portable medical imaging device of clause 29, wherein the image model for the possible images in the imaging domain comprises a model simulated using finite difference time domain (FDTD) to capture all significant electromagnetic paths and simulate effects of refractive/reflective interfaces within the imaging domain.

Clause 31: The portable medical imaging device of any of clauses 1-30, wherein the at least one controller is configured to auto-calibrate in order to remove effects within electronics and up to a skull of the patient from detected measurements made by the device.

Clause 32: The portable medical imaging device of clause 31, wherein auto-calibration is based on measurements for one or more propagation paths between pairs of the plurality of USB transceivers that occur irrespective of a presence of the patient's head in the interior of the housing.

Clause 33: The portable medical imaging device of clause 32, wherein the one or more propagation paths for calibration are captured in a data set along with signals for paths between pairs of the plurality of UWB transceivers used for generating the image.

Clause 34: The portable medical imaging device of clause 32 or clause 33, wherein the one or more propagation paths used for auto-calibration are not used for generating the medical image.

Clause 35: The portable medical imaging device of any of clauses 1-34, wherein the at least one controller is further configured to apply filter processes to the received and processed information and generate reconstructed medical images based on the filtered information in which areas meeting particular criteria are highlighted or otherwise identified.

Clause 36: The portable medical imaging device of clause 35, wherein highlighting or identification is based on identified regions having substantial changes in relative permittivity compared to other areas of the generated images.

Clause 37: The portable medical imaging device of clause 35 or clause 36, where the regions meeting particular criteria comprise regions evidencing right/left differences between hemispheres of the brain, blood or blood depleted regions, and/or candidate stroke regions.

Clause 38: The portable medical imaging device of any of clauses 35-37, wherein the filtering process by the at least one controller comprises analyzing the generated image based on previously obtained MRI/CT to RF comparison data in order to identify areas in the generated medical image corresponding to physician segmented areas identified in a prior MRI/CT dataset.

Clause 39: The portable medical imaging device of any of clauses 1-38, wherein the at least one controller is configured to generate the medical images by comparing the received and processed information comprising measured time-of-flight data for time-of-flight between antenna pairs of the plurality of UWB transmitters to a pre-calculated model comprising all conceivable images and their relation to impulse responses across the antenna pairs.

Clause 40: The portable medical imaging device of clause 38, wherein the pre-calculated model comprise a model generated by DTD/FE prediction tools executed across a voxel representation of a 3D space for all possible relative permittivities of the voxel.

Clause 41: The portable medical imaging device of any of clauses 1-40, further comprising at least one location beacon configured to transmit radio frequency signals indicating a location of the device.

Clause 42: The portable medical imaging device of clause 41, wherein the at least one location beacon comprises: at least one cellular transmitter for transmission of wireless signals via a cellular network; and at least one ultra-wide band (UWB) transmitter for transmission of radio signals over a short-range network.

Clause 43: The portable medical imaging device of clause 41 or clause 42, wherein the at least one controller is configured to periodically cause the at least one location beacon to communicate location and status information for the device via the at least one cellular transmitter or the at least one UWB transmitter.

Clause 44: The portable medical imaging device of clause 42 or clause 43, further comprising a battery for the at least one location beacon configured to support at least ˜500 cellular blinks and/or about 5000 UWB blinks prior to depletion of the battery.

Clause 45: The portable medical imaging device of any of clauses 42-44, wherein the at least one controller is configured to receive location information based on signals received by the UWB transmitter and to cease communication via the cellular transmitter when location information indicates that the device is within a facility including a UWB gateway.

Clause 46: The portable medical imaging device of clause 45, wherein the at least one controller is configured to reduce a blink rate of the at least one location beacon when the device is outside of a locationing facility.

Clause 47: The portable medical imaging device of clause 45 or clause 46, wherein the at least one controller is configured to increase a blink rate of the UWB transmitter when the device is inside the facility or proximate to a portable electronic device (e.g., a smart phone) equipped to locate the UWB emitter.

Clause 48: The portable medical imaging device of any of clauses 44-47, wherein the at least one controller is configured to apportion battery power from the battery by radio type providing about 90% for the cellular transmitter and less than about 10% to the UWB transmitter.

Clause 49: The portable medical imaging device of any of clauses 45-48, wherein the communication information can further comprise location information provided by at least one of Wi-Fi sniffing, a cellular E-911 network, the UWB transmitter, a global positioning network or similar capability available from a communication connection such a Wi-Fi or UWB with a vehicle telemetry hub.

Clause 50: The portable medical imaging device of any of clauses 42-49, further comprising at least one environmental sensor disposed on the housing and configured for detection of at least one of temperature, humidity, shock, and/or vibration of or proximate to the device.

Clause 51: The portable medical imaging device of clause 50, wherein the at least one environmental sensor is configured to detect acceleration and temperature information for the device, and wherein the at least one controller is configured to modify power usage of the at least one cellular transmitter and/or the at least one UWB transmitter based on the detected acceleration and temperature information.

Clause 52: The portable medical imaging device of clause 51, wherein the at least one controller is configured to increase a blink rate for one or more of the at least one cellular transmitter or the at least one UWB transmitter upon a detected change in the acceleration or temperature information.

Clause 53: The portable medical imaging device of any of clauses 42-52, wherein the cellular transmitter supports at least the following communication standards: NB-IoT, LTE-M, and CATM.

Clause 54: The portable medical imaging device of any of clauses 1-53, wherein heat generated by the plurality of UWB transmitters and/or the patient is removed from the device conductively through the housing.

Clause 55: The portable medical imaging device of any of clauses 1-54, further comprising a battery and a plurality of cables extending through the housing for providing power from the battery to the plurality of transmitters.

Clause 56: The portable medical imaging device of clause 55, wherein the cables comprise a cable harness mounted to interior surfaces of the housing.

Clause 57: The portable medical imaging device of clause 55 or clause 56, wherein the battery is modular and capable of being removed from the device and replaced.

Clause 58: The portable medical imaging device of any of clauses 55-57, wherein the battery supports image generation operation of the device for up to at least one day.

Clause 59: The portable medical imaging device of any of clauses 55-58, wherein the battery comprises a non-rechargeable single use battery.

Clause 60: The portable medical imaging device of any of clauses 1-59, further comprising a wireless data transceiver configured to wirelessly transmit received and processed information collected by the device to a remote computing device and/or computer network.

Clause 61: The portable medical imaging device of clause 60, further comprising an activation button configured to cause the wireless data transceiver to initiate transmission of the received and processed information to the remote computing device and/or computer network.

Clause 62: A medical imaging system, comprising at least one of the portable medical imaging devices of any of clauses 1-61; a wireless transmitter in communication with the at least one controller of the at least one portable medical imaging device; and at least one remote computing device configured to wirelessly receive data from the at least one portable medical imaging device and to generate one or more medical images based on measurement data wirelessly received from the at least one portable medical imaging device.

Clause 63: The system of clause 62, further comprising a feedback device configured to display the generated at least one medical image.

Clause 64: The system of clause 63, wherein the feedback device comprises an augmented reality device.

Clause 65: The system of any of clauses 62-64, wherein the remote computing device is configured to transmit the generated one or more medical images to a computer network allowing remote devices to access the one or more medical images via the computer network.

Clause 66: The system of any of clauses 62-65, wherein the remote computing device comprises a portable computing device or smart phone in communication with the at least one device via the wireless transmitter.

Clause 67: A portable medical imaging device, comprising: a blanket comprising an outwardly facing surface and an inwardly facing surface configured to be positioned proximate to a patient; a radio frequency absorbing material positioned in the blanket between the inwardly facing surface and the outwardly facing surface of the blanket; a radio frequency matching liquid configured to be positioned between the radio frequency absorbing material and the patient, while the patient is covered by the blanket; a radio frequency array comprising a plurality of ultra-wide band (UWB) transceivers distributed in the radio frequency absorbing material configured to direct radio frequency signals to the patient and to detect radio frequency signals emitted by other transceivers of the plurality of UWB transceivers; and at least one controller configured to receive and process information about signals detected by the plurality of UWB transceivers to generate medical images.

Clause 68: The portable imaging device of clause 67, wherein the blanket comprises a first portion configured to be positioned over the patient and a second portion configured to be positioned under the patient, such that the patient is wrapped within the blanket.

Clause 69: The potable imaging device of clause 67 or clause 68, wherein the blanket is configured to cover an upper portion of the patient's body.

Clause 70: The portable imaging device of any of clauses 67-69, wherein the blanket is configured to cover the patient's whole body.

Clause 71: A tracking and data collection system for medical imaging, the system comprising: a plurality of the portable medical imaging devices of any of clauses 1-61; and a computer server configured to receive and process signals communicated from the plurality of imaging devices, determine a location for one or more of the plurality of portable medical imaging device based on the received and processed signals, generate medical images based on the received and processed signals, and store generated images in electronic medical records associated with one or more of a plurality of patients.

Clause 72: A method of treatment for a patient suspected of having and/or diagnosed with Alzheimer's disease, comprising: capturing at least one medical image of the patient using the portable medical imaging device of any of clauses 1-61; analyzing the captured at least one medical image to determine a characteristic of Alzheimer's tissue visible in the captured at least one medical image; and providing at least one medication at a dosage determined based, at least in part, on the analysis of the captured image.

Clause 73: The method of clause 72, wherein analysis of the captured at least one medical image identifies Amyloid-Related Imaging Abnormalities (ARIA) in the captured at least one medical image.

Clause 74: The method of clause 72 or clause 73, wherein providing the at least one medication comprises administering a drug targeting amyloid beta plaques to the patient.

Clause 75: The method of clause 74, wherein the administered drug comprises lecanemab.

Clause 76: The method of clause 74 or clause 75, further comprising adjusting a dosage of the administered drug based on additional medical images generated by the at least one portable medical imaging device based on measurement data captured at predetermined intervals.

Clause 77: A method of identifying tissues indicative of traumatic brain injury, comprising: capturing at least one medical image of a head of the patient using the portable medical imaging device of any of clauses 1-61; and analyzing the captured at least one medical image to identify areas of the at least one captured image evidencing one or more characteristics of traumatic brain injury.

Clause 78: A method of triage for a patient who has suffered and/or is suffering a stroke, comprising: capturing at least one medical image of a head of the patient using the portable medical imaging device of any of clauses 1-61; analyzing the captured at least one medical image to distinguish between areas of the at least one captured image evidencing an ischemic stroke and a hemorrhagic stroke; and treating the patient for either the identified ischemic stroke or the identified hemorrhagic stroke.

Clause 79: The method of clause 78, wherein treating the patient for ischemic stroke comprises monitoring the patient by obtaining additional medical images with the portable medical imaging device once per day to identify relapse and/or hemorrhagic transformation.

Clause 80: The method of clause 78 or clause 79, wherein treating the patient for hemorrhagic stroke comprises monitoring the patient once per hour for between 24 and 48 hours following identification of the hemorrhagic stroke and providing blood pressure control for the patient.

### BRIEF DESCRIPTION OF THE DRAWINGS

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only, and are not intended as a definition of the limit of the invention.

FIG. 1 is a schematic drawing of a medical imaging device, according to an aspect of the present disclosure;

FIGS. 2A-2C are additional schematic drawings showing cross-sectional views of medical imaging devices, according to aspects of the present disclosure;

FIG. 3 is a schematic drawing showing electronic components of a medical imaging device, according to an aspect of the present disclosure;

FIG. 4A is a side view of a medical imaging device embedded within a clam-shell helmet, according to an aspect of the present disclosure;

FIG. 4B is a front view of the helmet of FIG. 4A worn by a patient;

FIG. 4C is a front view of another example of a medical imaging device embedded within a clam-shell helmet, according to an aspect of the present disclosure;

FIG. 5A is a schematic drawing of a front view of medical imaging device comprising an upper body blanket, according to an aspect of the present disclosure;

FIG. 5B is a drawing of the medical imaging device of FIG. 5A on a patient, according to an aspect of the present disclosure;

FIG. 5C is a schematic drawing showing a cross-sectional view and electronic components of the medical imaging device of FIG. 5A;

FIG. 6A is a schematic drawing of a front view of a medical imaging device comprising a whole body blanket, according to an aspect of the present disclosure;

FIG. 6B is drawing of the medical imaging device of FIG. 6A on a patient, according to an aspect of the present disclosure;

FIGS. 7A and 7B are a flow chart showing an image processing method for generating medical images, according to an aspect of the present disclosure; and

FIG. 8 is a schematic drawing of a cloud-based medical imaging system including multiple medical imaging devices, according to an aspect of the present disclosure.

### DESCRIPTION OF THE INVENTION

As used herein, the singular form of “a”, “an”, and “the” include plural referents unless the context clearly states otherwise.

As used herein, the terms “right”, “left”, “top”, “bottom”, and derivatives thereof shall relate to the invention as it is oriented in the drawing figures. However, it is to be understood that the invention can assume various alternative orientations and, accordingly, such terms are not to be considered as limiting. Also, it is to be understood that the invention can assume various alternative variations and stage sequences, except where expressly specified to the contrary. It is also to be understood that the specific devices and processes illustrated in the attached drawings, and described in the following specification, are examples. Hence, specific dimensions and other physical characteristics related to the embodiments disclosed herein are not to be considered as limiting.

For the purposes of this specification, unless otherwise indicated, all numbers expressing, for example, dimensions, physical characteristics, and so forth used in the specification and claims are to be understood as being modified in all instances by the term “about.” Unless indicated to the contrary, the numerical parameters set forth in the following specification and attached claims are approximations that can vary depending upon the desired properties sought to be obtained by the present invention. Notwithstanding that the numerical ranges and parameters setting forth the broad scope of the invention are approximations, the numerical values set forth in the specific examples are reported as precisely as possible. Any measured numerical value, however, may inherently contain certain errors resulting from the standard deviation found in their respective testing measurements.

As used herein, the terms “comprising,” “comprise” or “comprised,” and variations thereof, are meant to be open ended.

Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

With reference to the figures, the present disclosure is directed to imaging systems 110 and portable medical imaging devices 10 that use radio frequency (RF) signals, particularly low power RF signals, for medical imaging purposes. Beneficially, the medical imaging devices 10 do not use ionizing radiation, reducing harm to the patient and practitioners. Furthermore, the medical imaging devices 10 are highly portable allowing for fast and frequent imaging leading to better patient outcomes. In some examples, the medical imaging devices 10 disclosed herein provide instantaneous imaging, which can be sensitive to blood concentration, providing, for example, a stroke-type detector.

In some examples, the medical imaging devices 10 can be operable in any room or outdoors because there are no high power RF or magnetics. Also, the imaging devices 10 do not require a specialized table or support. Instead, images can be obtained while the patient reclines on a standard bed or chair. The imaging devices 10 and methods disclosed herein can be used for Alzheimer's screenings and/or ARIA monitoring, which can accelerate drug deployment compared to conventional imaging processes (e.g., CT/MRI) used for drug delivery. In one particular example, the imaging devices 10 disclosed herein can be used to screen entire population(s) for plaque deposits and other tissue abnormalities indicative of Alzheimer's disease decades before symptoms for Alzheimer's manifest in order to begin treatment for affected individuals far earlier than currently occurs. Also, obtained images can be used to detect ARIA during treatment, which dramatically lowers cost of treatment by virtually eliminating the high cost of companion MRI.

In some examples, the imaging devices 10 disclosed herein provide a unique 3D millimetric imaging design, commensurate with CT/MRI, that nonetheless also provides technical superiority to fundamental limitations in current CT/MRI solutions. In examples, the imaging devices 10 can resemble a helmet-like head unit. In other examples, body scanning units akin to blankets can also be provided. The head units or blankets can be used in a medical facility, at home, or in mobile vehicles, such as air/space craft or ambulances. The imaging devices 10 disclosed herein desirably address core pain points in medical imaging, such as the balance between diagnostic benefit, patient harm, and cost. By eliminating patient risk, offering rapid/early use, superior tissue contrast and lesion conspicuity, and broad DICOM/Augmented Reality compatibility and reconstructions augmented with deep learning, the imaging devices 10 promote healthcare equity and decrease healthcare costs.

In some examples, the imaging devices 10 disclosed herein can be configured for direct radio-wave propagation to provide medical imaging at clinically relevant resolutions. The dynamic range of relative permittivity of tissue promotes imaging conspicuity at clinically relevant resolution while dramatically reducing cost, consequently increasing availability and utilization, expanding capability beyond the diagnostics center.

In some examples, the imaging devices 10 comprise a single person hand-carryable three-dimensional radio frequency (3D RF) tomographer with millimetric resolution and near instantaneous image generation. It is believed that such a portable device provides dramatic cost reduction and increased access for medical imaging compared to current MRI/CT imaging options. Therefore, it is believed that medical imaging provided by the imaging devices 10 disclosed herein will substantially improve access to medical imaging proving crucial for early detection and monitoring of disease and therapy progression, enabling early clinically actionable therapy.

In some examples, a carryable 3D imager, such as the imaging devices 10 disclosed herein, can be deployed in the home or low acuity care facility, which may reduce missed appointments, doubling healthcare resource efficiency. It is believed that an RF tomography device's sensitivity to Alzheimer's tissue allows for detailed tracking of disease progression and effective screening. Also, imaging provided by the imaging devices 10 disclosed herein is clean, inherently safe, electronically steered (no moving parts), and non-invasive, which eliminates risks associated with traditional diagnostic imaging procedures (requiring ionizing radiation, high power RF/magnetic fields and contrast agents), enabling repeated screening and monitoring use.

In some examples, the imaging devices 10 disclosed herein use multiple (e.g., from 100 to 1000 or more) ultra-wideband antennas operating, for example, at a frequency of 3.0 GHz+ (e.g., at a frequency of between about 3 GHz and about 8 GHz). By contrast, existing RF imaging devices may operate at about a single tone frequency of 1 GHz or less. It is believed that the imaging devices 10 and associated systems 110 disclosed herein provide cost-effective UWB technology, sidestepping the need for complex switching and control, and enabling wide-scale deployment for transformative medical imaging.

In some examples, the portable medical imaging devices 10 use impulse responses for the UWB sensors (e.g., UWB antenna or transceivers) for imaging purposes. As described in further detail herein, certain impulse responses will contain returns from paths that are not wanted (such as through the rear of the antenna and around the inside of the helmet). Such unwanted impulse responses may potentially be excised using filters. In some examples, the pulses emitted by UWB transceivers can be coded pulses unique to each UWB transceiver of an RF array. In this way, it is possible to determine where any detected RF signal originated from. Each receiver of the UWB transceiver can also be configured to decode the coded pulse and associate a time of reception with the pulse. In some examples, the imaging devices 10 can be configured to generate a single first time and power that can be used to establish free space distance. The imaging devices 10 are also capable of providing a range profile, where each receiver measures power for each UWB transceiver over time. The measurements can be analyzed to define material distribution in space within an imaging domain. In some examples, time is also changed by the medium through which propagation occurs. Thus, unlike in the free space case of locationing, there is also a possibility that signal(s) take longer to arrive than the speed of light would suggest. Particularly in brain tissue, the speed of light is slowed down for these RF signals by around 7×meaning that detected tissue seems larger than expected.

As previously described, the medical imaging devices 10 can include an RF transmitter. The RF transmitter can be similar or identical to transmitters used for UWB tags (e.g., Apple AirTags), which are arranged in a mesh configuration creating a measurement network for imaging. Furthermore, the portable (e.g., hand carry-able) imaging device 10 can be user-friendly requiring no specialized training. In addition, remote physician support can be provided over, for example, a computer network, Wi-Fi, a cellular network, or the Internet from the cloud helping to ensure accurate deployment, operation, and interpretation.

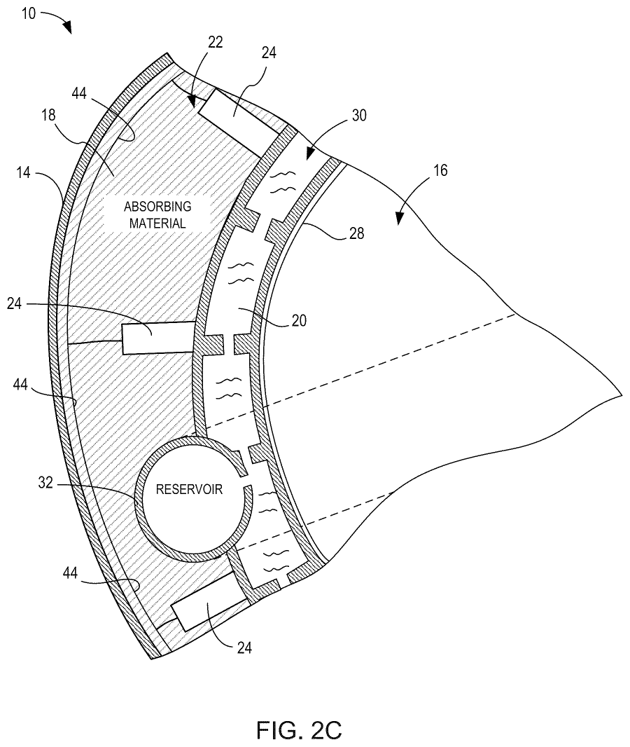

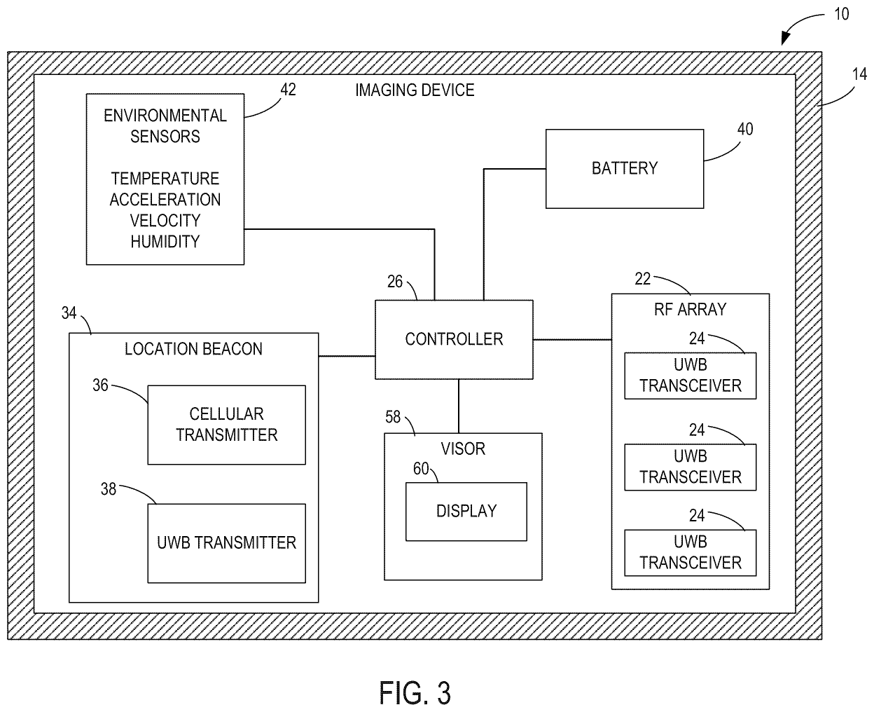

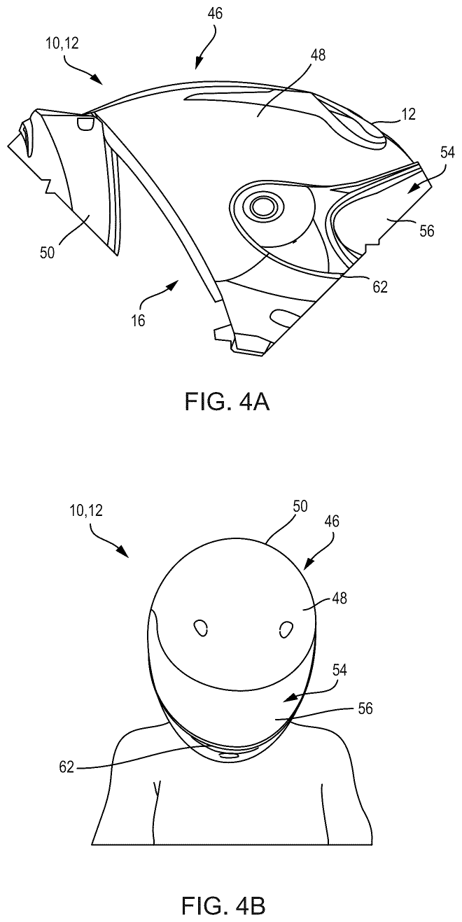

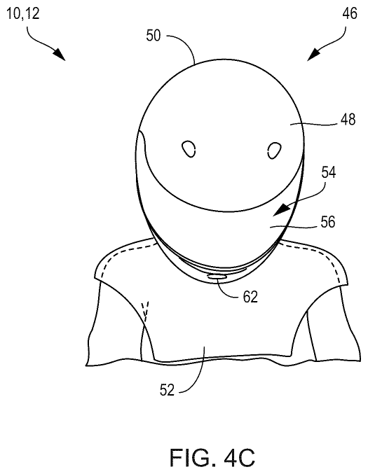

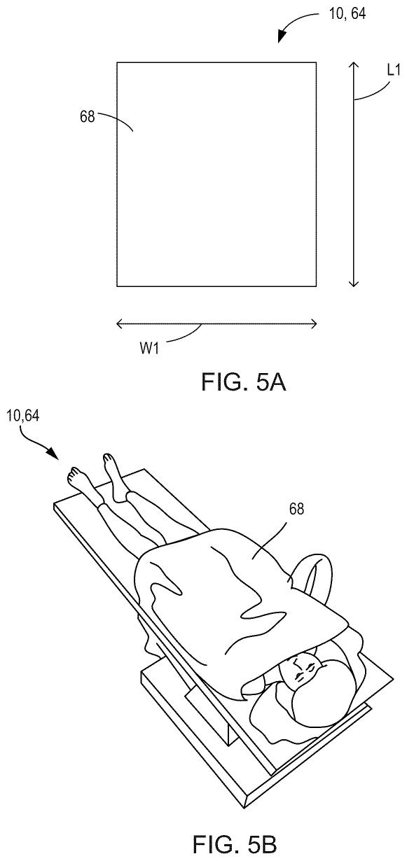

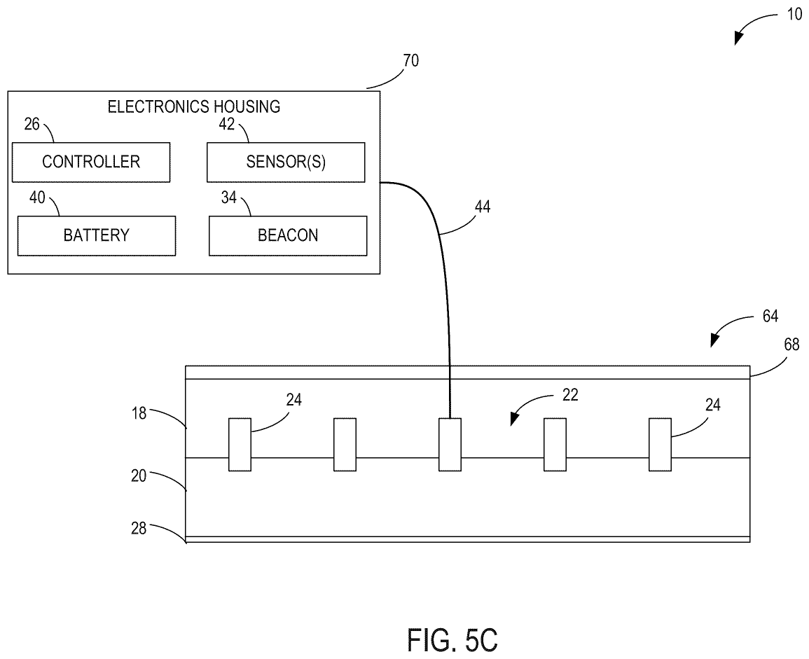

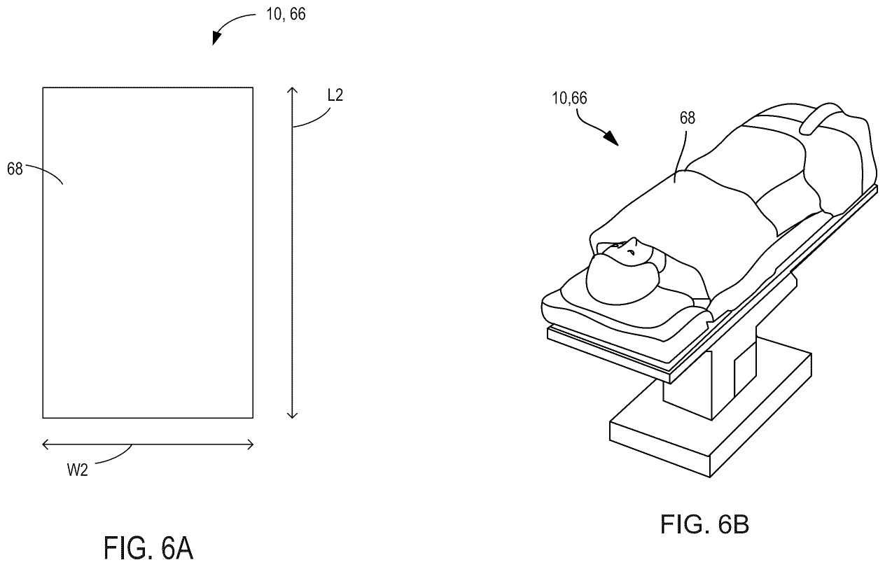

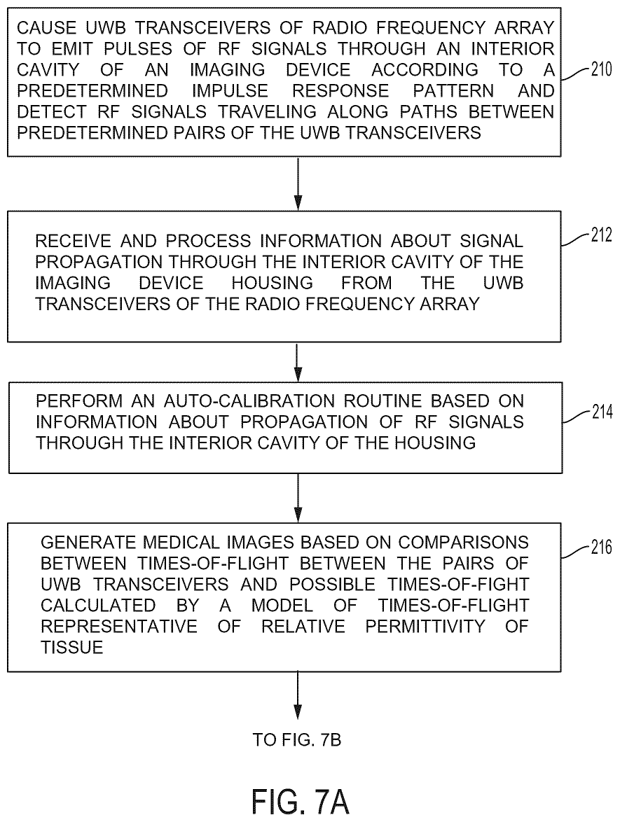

In some examples, the portable medical imaging devices 10 comprise or are enclosed within a helmet 12 (e.g., a crash helmet). An example of a crash helmet 12 for the medical imaging device 10 is shown in FIGS. 4A-4C. In other examples, components of the portable medical imaging device can be embedded in a blanket 64, 66, as shown in FIGS. 5A-6B. As previously discussed, the imaging devices 10 can be capable of real-time operation, which is believed to be possible for an imaging device 10 that provides 1 million UWB measurement cycles at 10 Hz.

In some examples, the imaging device 10 can be used with systems 110 for remote or cloud processing. Beneficially, the small measurement vector size, conducive to low bandwidth cloud communications, ensures usability even in rural areas, extending equitable reach of the imaging device 10. In particular, a remote computer server accessed via the cloud can process each vector for its specific medical application, making the medical imaging device 10 a versatile screening tool for various medical conditions, such as Alzheimer's, stroke, and cancer.

In some examples, the imaging devices 10 can be deployed in public locations (e.g., government buildings, corporate offices, theaters, sporting venues, etc.), similar to how AED units are currently deployed. When widely available, the imaging devices 10 can be used by emergency/rescue personnel to, for example, identify traumatic brain injury, strokes, and similar conditions in order to quickly provide appropriate treatment in emergency cases. The patient images and early identification of condition (e.g., traumatic brain injury or stroke) can also ensure that a patient is delivered to a facility that is best able to provide appropriate treatment for identified conditions. By ensuring that a patient is initially transported to a correct facility, time lost during triaging the patient and/or transporting the patient to another facility after initial examination can be substantially reduced.

### Portable Medical Imaging Devices

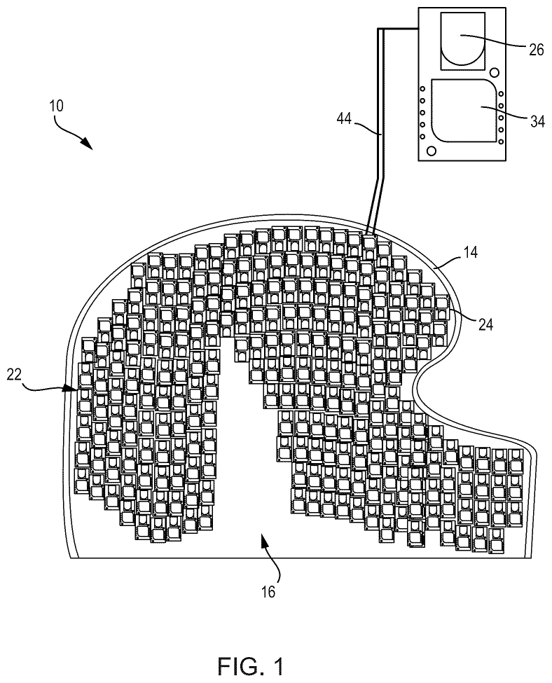

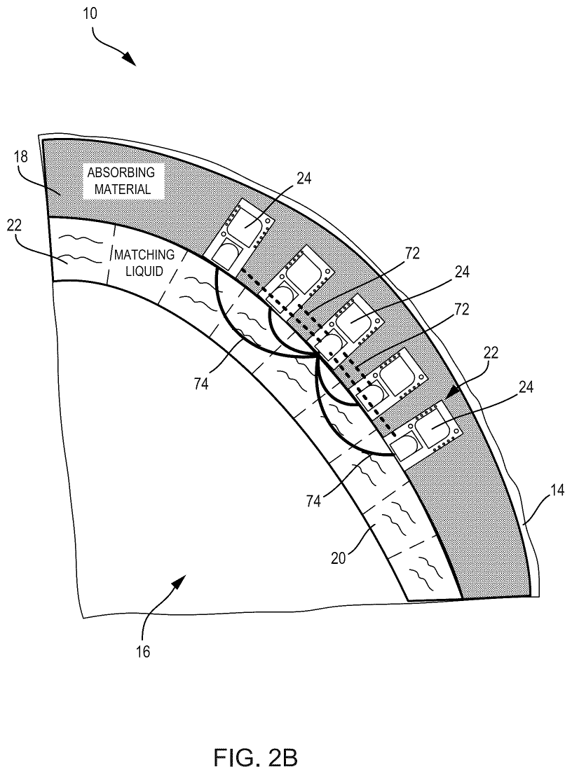

Features of the portable imaging device 10 are shown in FIGS. 1-3. In some examples, the portable imaging device 10 comprises a housing 14 defining an interior cavity 16 sized to receive a head of a patient. The portable medical imaging device 10 further comprises a radio frequency absorbing material 18 positioned in the interior cavity 16 of the housing 14 and a radio frequency matching liquid 20 configured to be positioned between the radio frequency absorbing material 18 and a patient's head received within the interior cavity 16 of the housing 14. The portable medical imaging device 10 further comprises a radio frequency array 22 comprising multiple ultra-wide band (UWB) transceivers 24 distributed in the radio frequency absorbing material 18 configured to direct radio frequency signals towards the patient's head and to detect radio frequency signals emitted by others of the UWB transceivers 24, which have passed through patient (e.g., brain) tissue. In addition, the medical imaging device 10 further comprises a computer processor or controller 26 (shown in FIGS. 1 and 3) configured to receive and process information about the radio frequency signals detected by the multiple UWB transceivers 24 for generation of medical images of the patient's head. As previously described, the portable imaging device 10 and housing 14 are configured to be lightweight and comfortable enough to be worn by the patient for a period of time sufficient to obtain multiple medical images. For example, the medical imaging device 10 including the housing 14 and other electronical components can be less than about 50 pounds, less than about 40 pounds, or preferably about 36 pounds. In some cases, time needed to obtain medical image data can be as short as a few seconds, while in other cases (e.g., for a conscious patient undergoing a more comprehensive image study) wearing the device 10 for up to ten minutes or longer may be possible. In some examples, the imaging device 10 can also be worn continuously allowing images to be captured periodically or according to a predetermined schedule. For example, an unconscious stroke patient may wear the imaging device 10 for several hours or up to several days, allowing for consistent monitoring over a period of time to identify changes in patient status or condition.

As previously described, the portable medical imaging device 10 is configured to emit RF waves or signals within ultra-wide band (UWB) frequencies for medical imaging via the UWB transceivers 24. UWB ranging transformation utilizes UWB ranging to model electrical properties (e.g., measuring time and power) of the imaging domain. Measured electrical properties are correlated with a finite difference time domain impulse response model through the fixed distance in which propagation occurs. By correlating time and power measurements in this way, rather than by assuming free space and calculating node separation, the imaging devices 10, in effect, reverse conventional uses for time-of-flight UWB emitters and detectors, determining electrical properties of the media between antennas separated by fixed locations rather than determining separations between antennas for fixed media (e.g., free space).

In an example, the imaging device 10 comprises a 300 element array with 300 UWB nodes, deployed inside a helmet (shown in FIGS. 4A-4C). The 300 elements (e.g., UWB transceivers 24) and associated nodes can operate in an impulse response measurement mode. Because the measurement vector is a matrix of impulse response measurements, each will need control, but all may need reception within 0.1 sec. In some instances, this interval may be divided into 1,000 non-interfering transmissions slots and every receiver configured to deliver its impulse response for every transmitter case. Also, in some examples, the UWB transceivers 24 can operate with a bandwidth of at least 500 MHz. In other examples, the UWB transceivers 24 operate at a frequency of about 3 GHz to about 8 GHz or about 3.5 GHz to about 9 GHz. This frequency improves resolution and also saves weight compared to lower operating frequency transceivers (e.g., transceivers operating about 1 GHz) because the matching liquid 20 and RF absorbing material 18 of the imaging device 10 can be commensurately thinner. Because each UWB transceiver 24 both transmits and receives RF signals, an image generated from an array of 300 UWB transceivers 24 produces an image based on 300×300 measurements. In other examples, the RF array 22 can comprise 1,0000 UWB transceivers 24 or more. An image generated from an array comprising 1,000 UWB transceivers 24 is based on 1,000×1,000 measurements.

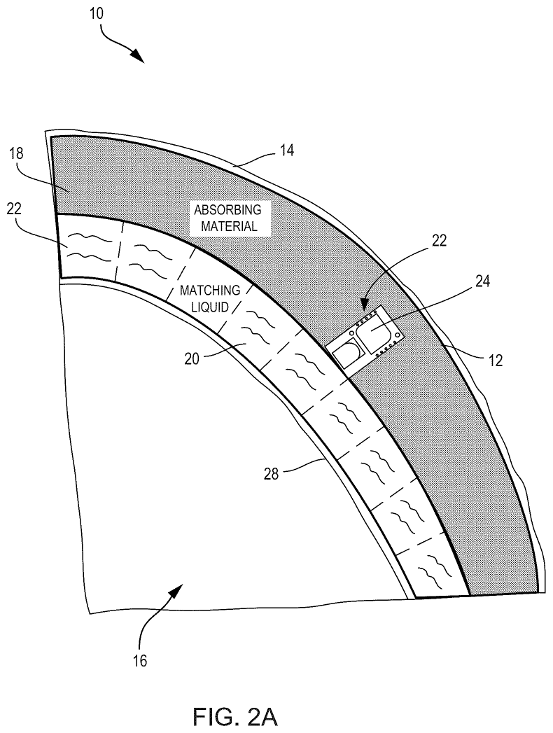

As shown in FIGS. 2A-3, the radio frequency array 22 of UWB transceivers 24 can be distributed in the radio frequency absorbing material 18 inside the housing 14 with the patient protected by a membrane 28 enclosing the matching liquid 20. In addition to supporting the USB transceivers 24, the absorbing material 18 can also be configured to function as an absorber behind the UWB transceivers 24 in order to damp reflections from, for example, a helmet shell into the imaging domain. In some examples, to ensure propagation of radio frequency energy from the UWB transceivers 24 into the brain, the UWB transceivers 24 must be “matched” in radio frequency terms to relative permittivity of patient tissue (e.g., the brain). The “matching” can be achieved by filling gaps between the patient and the UWB transceivers 24 with the matching liquid 20 and specifically pushing out any air, which becomes a disrupter to measurement correlation. Thus, the matching liquid 20 can be selected to have a permittivity configured to match brain permittivity and positioned to avoid air gaps between the plurality of UWB transceivers 24 and the patient. Rather than outside the metal chamber, the UWB transceivers 24 of the radio frequency array 22 can be embedded within the absorbing layer or RF absorbing material 18 allowing the imaging device 10 to become thinner and lighter. While the UWB transceivers 24 are primarily held in the generally solid radio frequency absorbing material 18, the UWB transceivers 24 can also protrude slightly into the matching liquid 20 meaning that the housing 14 can be thinner compared to examples where the UWB transceivers 24 are entirely separate from the matching liquid 20. In some examples, reflections inside the housing 14 emitted from the USB transceivers 24 are separated by impulse response. Also, multiple internal reflections can be damped from a predicted value by the absorption in the RF absorbing material 18 and the matching liquid 20, speeding up processing. In particular, the damped cases tend to fall off rapidly and not matter much given the plurality of receivers involved in capturing key aspects of an image, namely tissue and/or materials of clinical interest and their disposition in the brain.

In some examples, the membrane 28 containing the matching liquid 20 can be a flexible polymeric material. In some examples, the thin layer of the matching liquid 20 can become conformal to each individual patient's head size and shape by creating a compressible celled support structure 30 (shown in FIG. 2C), such as a honeycomb liquid filled structure, as shown most clearly in FIG. 2C. For example, the RF absorbing material 18 into which the UWB transceivers 24 are embedded can be fronted by liquid filled rubber cubes of the celled support structure 30 with wall holes that allow the matching liquid 20 to freely move between cubes. The material of the celled support structure 30 can be doped rubber or another material electromagnetically similar to rubber. The weight, mass, or volume of the patient's head, along with compressive forces produced by closing the housing 14 or helmet 12 around the head, can be configured to force the matching liquid 20 to become conformal to the patient's entire head, driving out air pockets that can be disruptive to images.

In some examples, the compressible celled support structure 30 comprises multiple cells containing the matching liquid 20 and a liquid reservoir 32 (shown in FIG. 2C) in fluid communication with the multiple cells configured so that the matching liquid 20 passes between the multiple cells and the reservoir 32 to accommodate patient heads of different sizes. In some examples, the liquid reservoir 32 can become a donut-shaped reservoir around the outside of the housing 14. Also, in some examples, the celled support structure 30 can be formed from rubberized walls configured to deform when the patient's head is inserted into the interior cavity 16 of the housing 14. In this way, the celled support structure 30 can be configured to allow larger heads to displace more matching liquid 20 into the reservoir 32. Larger heads also crush or deform portions of the celled support structure 30 uniformly about the head, resulting in uniform distribution of the matching liquid 20. By a simple modification to, for example, a volume of the matching liquid 20 and/or configuration of the celled support structure 30, a variant of the housing 14 can also be configured to support the pediatric patient population where perhaps the traumatic brain injury application might become more relevant.

In the imaging devices 10 disclosed herein, the UWB transceivers 24 are configured to measure RF propagation of impulse response through body (e.g., brain) tissue to determine constituent material i.e. to generate an image. Examples of impulse response paths 72 and optical calibration paths 74 between pairs of UWB transceivers 24 are shown in FIG. 2B. The imaging techniques disclosed herein may be a reverse of the calculation used to determine location of an active tag (e.g., an AirTag) in free space, where time translates through speed of light to separation. In examples, the UWB transceivers 24 (e.g., UWB time-of-flight silicon transceivers, similar or identical to transceivers used by Apple AirTags for locationing), can be configured to enable 10× node growth permitting higher frequency and 4× resolution improvement over competitive devices.

In some examples, the radio frequency array 22 of the medical imaging device 10 creates a fixed mesh around an imaging domain (fixed separation) and measures time-of-flight in order to determine materials through which propagation has occurred. The time-of-flight measurements are also representative of the paths through those materials along which the RF signals travel. Thus, for example, reflection and diffraction may yield more than one time-of-flight measurement between transmit and receive, as measured by one or more of the UWB transceivers 24. Therefore, similar to bi-static radar, peaks may be observed in a correlation of the transmitted signal starting at the shortest possible time to traverse space between the transmitter and receiver. Other peaks may be observed where some of the RF energy takes an indirect path between the transmitter and receiver resulting in a range profile. Material which slows down or interrupts speed at which propagated signals transmit through space and/or loss of signal power are directly related to relative permittivity. Therefore, the imaging devices 10 and methods disclosed herein can use detected time-of-flight and/or loss of power measurements for generating images of distribution of relative permittivities that vary by tissue type, especially between healthy and unhealthy tissue types, such as normal brain tissue and amyloid and tau tangle affected Alzheimer's tissue, or blood depleted (ischemic) tissue and blood hemorrhages themselves.

### Communication and Location Detection

FIG. 3 is a schematic drawing showing electrical components of the imaging device 10. As shown in FIG. 3, in some examples, the imaging device 10 can include wireless communication, location determining, and/or tracking circuitry for monitoring location of one or more imaging devices 10 prior to and during use. Communication circuitry can be used for wirelessly transmitting measured information and/or generated images from the imaging device 10 to a remote computing device or computer network. Location information for imaging devices 10 may be used to coordinate device delivery or deployment, allowing devices to be delivered to a patient's home or workplace for obtaining medical images away from medical facilities. Location information may also be used to coordinate delivery service activities, such as planning delivery routes to ensure efficient use of imaging devices 10. For example, location information for imaging devices 10 may be used to confirm that an imaging device 10 is delivered to a desired location at an appropriate time for performing a scheduled imaging procedure.

In order to provide such communication, location, and/or tracking information, the imaging device 10 can further comprise a communication and location beacon 34 configured to transmit radio frequency signals with imaging information and/or indicating the location of the imaging device 10 to remote devices and/or networks. To promote efficient data transfer and power efficiency, the communication and location beacon 34 can comprise multiple communication options or transmitters. For example, the communication and location beacon 34 can comprise a cellular transmitter 36 for transmission of wireless signals via a cellular network and an ultra-wide band (UWB) transmitter 38 for transmission of radio signals over a short-range network. To provide location determination, the controller 26 of the imaging device 10 can be configured to periodically cause the communication and location beacon 34 to communicate location and status information for the imaging device 10 via the cellular transmitter 36 and/the UWB transmitter 38.

In some examples, the imaging device 10 further comprises one or more batteries 40 for providing power to the transmitters 36, 38 for data transmission. In some examples, the one or more batteries 40 can be configured to support at least ˜500 cellular blinks and/or about 5000 UWB blinks prior to depletion of the battery.

In some examples, the controller 26 can also be configured to control the communication and location beacon 34 to transmit data with maximum efficiency and/or using minimal power to conserve battery life. For example, the controller 26 can be configured to receive location information based on signals received by the UWB transmitter 38 and to cease communication via the cellular transmitter 36 when location information indicates that the imaging device 10 is within range of a suitable UWB gateway device, such as within a medical facility including UWB gateway devices installed throughout the facility. Similarly, the device controller 26 can be configured to reduce a blink rate of the communication and location beacon 34 when the imaging device 10 is outside of the facility and/or to increase a blink rate of the UWB transmitter 38 when the imaging device 10 is inside the facility and in proximity to one of the UWB gateway devices. In some examples, the device controller 26 can also be configured to apportion battery power from the battery 40 by radio type providing, for example, at least 90% power for the cellular transmitter 36 and less than about 10% power for the UWB transmitter 38.

In some examples, the imaging device 10 can also be configured to receive location information from other location determining networks and/or other devices in communication with the location determining circuitry or beacon 34 of the imaging device 10. For example, the imaging device 10 can be configured to receive location information provided by Wi-Fi sniffing, a cellular E-911 network, or a global positioning network associated with, for example, a vehicle used for transporting the imaging device 10 to a location for medical use. As described in further detail herein in connection with remote monitoring systems 110, the controller 26 of the imaging device 10 can also be configured to transmit detected location information to a remote computing device and/or computer network for review and consideration by others.

In some examples, as shown in FIG. 3, the imaging device 10 can further comprise environmental sensors 42 for detecting a condition of and/or conditions proximate to the imaging device 10. Detected environmental information can be used for determining a status of the imaging device 10 and/or whether the imaging device 10 may have been mistreated in a way that potentially reduces accuracy of RF signals detected by the radio frequency array 22 of the imaging device 10 and/or which may damage any other components of the imaging device 10.

In some examples, the environmental sensor 42 can be disposed on the housing 14 of the imaging device 10 and configured for detection of one or more of temperature, humidity, shock, and/or vibration of or proximate to the imaging device 10. Information detected by the environmental sensor 42 can be used for controlling the imaging device 10 and/or for assessing device status. For example, the environmental sensor 42 can be an accelerometer for detecting movement of the imaging device 10 as may occur if the imaging device 10 is misused or mistreated and/or a temperature sensor for detecting if, for example, the imaging device 10 is exposed to extreme temperatures, which could damage device electronics or affect accuracy of collected imaging data. Also, in some examples, the device controller 26 can be configured to modify power usage of the cellular transmitter 36 and/or UWB transmitter 38 based on the detected acceleration and temperature information. Similarly, the device controller 26 can be configured to increase a blink rate for the cellular transmitter 36 or the UWB transmitter 38 upon a detected change in the acceleration or temperature information.

As previously described, the imaging device 10 further comprises one or more of the batteries 40 for providing power to the imaging electronics, such as the multiple UWB transceivers 24 of the radio frequency array 22, and/or for providing power for communications and location determining circuitry of the imaging device 10. Desirably, the battery 40 contains enough power to operate the imaging device 10 for a reasonable period of time (e.g., a standard eight hour workday) so that medical images can be obtained regardless of whether external power is available. Alternatively, the battery 40 can provide power for non-imaging functions of the imaging device 10, such as device status monitoring, location determination, and/or wireless data transfer, while external power can be used for obtaining medical images. For example, a user may plug the imaging device 10 into a wall outlet or another power source when the imaging device 10 is used to capture medical images of a patient.

In some examples, the imaging device 10 can comprise two or more separate batteries 40 or battery reservoirs. For example, a first battery 40 or battery reservoir can provide power for imaging actions of the imaging device 10. Desirably, this battery 40 lasts for at least a duration of a medical facility shift (e.g., about 8 hours or one day). The second battery 40 or battery reservoir can provide location information allowing users to track the imaging device 10 when it is not being used to capture medical images. Desirably, this second battery 40 or reservoir lasts for many days, months, or years so that the imaging device 10 can continue to send location information meaning that a misplaced or lost imaging device 10 can be found before the second battery 40 is deleted.

Also, the imaging device 10 can include embedded cables 44 (shown in FIG. 2B) extending through the imaging device 10 for providing power from the battery 40 to the UWB transceivers 24 of the radio frequency array 22 and/or other electrical components of the imaging device 10. The cables 44 can comprise a harness mounted to the interior surface of the housing 14 and electrically connected to the UWB transceivers 24 of the radio frequency array 22. Heat generated from the imaging device 10 and/or cables 44 can be removed (i.e. conductively dissipated) to surfaces of the housing 14. In other examples, power can be provided from the battery 40 to the UWB transceivers 24 wirelessly through induction or using other power transfer mechanisms as are known in the art.

In some examples, the battery 40 of the medical imaging device 10 can be a modular battery, which can be removed from the imaging device 10 when depleted and replaced or recharged. In other examples, the battery 40 can be a single-use battery (i.e. for an emergency situation). In examples, the battery 40 can provide continuous operation in, for example, a diagnostic center. The imaging device 10 can also include a power distribution and control system, which can be integrated with and/or in communication with the modular battery.

In specific examples, a modular battery 40 configured for use with the medical imaging device 10 can be used for the following two purposes (i) to run the imaging device 10; and (ii) to find the imaging device 10 when it is not running and/or disconnected from a power source. When running the imaging device 10, the battery 40 can be configured to support a range of in-service lifetimes, but typically no longer than a single day shift between charges. The battery 40 can be recharged via a wall charger. In some examples, when the imaging device 10 remains unpowered for a period of time, it can periodically communicate its location and state via built in cellular access technology, such as the cellular transmitter 36. A device that can locate itself anywhere in the global environment for five years will enable designated employees (e.g., gig workers) to retrieve units even after the battery 40 is substantially depleted.

As previously described, the imaging device 10 can also include features for preserving battery life. For example, a primary battery saving mechanism can be to use geo-fence and mode control when a blink (e.g., sending a communication signal) is stimulated. For example, a blink can be activated when the imaging device 10 is moved. Movement can be detected by the environmental sensors 42, such as an accelerometer, of the imaging device 10. In other examples, a blink can be created in response to a location scan or to changes detected by a temperature sensor, battery low state, or timer. A rate for stimulated or created blinks can be determined by geo-fenced location or time. In some examples, the blink payloads are constructed from scan data and/or sensor data.

Power consumption control can also be used to extend battery life. For example, when the imaging device 10 is outside UWB zone (no precision location), power consumption can be reduced by reducing the blink rate of UWB functionality and increasing the blink rate of the cellular functionality. More specifically, when not at a “precision locate” facility, UWB blinks at a lower rate, which conserves power. Cellular blinks can only be stimulated when location needs an update and/or relatively infrequently to conserve battery. By contrast, when inside UWB coverage zone (precise location), power consumption can be reduced by reducing the blink rate of the cellular functionality, while increasing the blink rate of the UWB. In some examples, the communication blinks emitted by the imaging device 10 can include location information, either E-911, Wi-Fi sniff, or UWB. Also, in some examples, images obtained via an image study can be sent to a cloud server via whichever available communications method consumes the least battery, e.g. Wi-Fi (when available) or cellular, determined based on the imaging device 10 location.

In another example, a main battery 40 of the imaging device 10, if available and not depleted, can be used to periodically enable a GPS location capture, which would otherwise rapidly deplete a battery intended to be used solely for cellular and UWB locationing. Once the main battery 40 is deleted, the locationing battery can be used to power periodic cellular and/or UWB blinks, as previously described.

In a specific example, motion detected by the accelerometer can wake-up or reset a blink mode. In the “new” blink mode, the blink frequency can be set to a higher rate from hibernation. For example, in hibernation, blinks can occur for example 1/day, 1/week, 1/month, 1/quarter, or 1/year. Scans, timers, or sensor events can stimulate blink rate changes to more or less frequently deliver blinks, e.g. triggered by temperature excursion or accelerometer shock events. Scan images can also be packed into cellular/Wi-Fi or UWB blinks. In some examples, all proximate users (e.g., imaging devices 10 within a particular distance of a remote server) can be reported through blinks enabling authentication of authorized users or recording of unauthorized scans for later authorization. In some examples, all blink rates can be increased as power is harvested or when vehicle or main power is available (i.e., from a vehicle being used to transport the imaging device 10). If external power or large enough main battery is available, the medical imaging device 10 can also become a UWB anchor, which can be authenticated to proximate user smartphone without password entry.

In some implementations, exemplary specifications and/or requirements for the medical imaging device 10 can include one or more of the following: ability to deliver 4× resolution over proven unique 3D solution, eliminating an existing 2 minutes of latency and reducing weight by ˜2× compared to existing RF imaging devices; better than 5 mm detection resolution at 100% sensitivity and 97% specificity on hemorrhagic exclusion; millimetric resolution; zero latency; total weight of about 50 pounds (about 23 kg) or less, 40 pounds (about 18 kg) or less, or preferably 36 pounds (about 16 kg) or less. More particularly, the imaging device 10 is configured to be carried by a single (male or female) staff member of a medical facility. Current guidelines generally require that a two person lift team is needed to lift any object weighing more than 50 pounds. Generally, a single male staff member is permitted to carry objects weighing 50 pounds or less. A single female staff member is permitted to carry objects weighing 36 pounds or less. Therefore, an imaging device 10 weighing 36 pounds or less can be carried by a single male staff member or a single female staff member.

Additional features are listed in the following table.

| Feature | Requirement |
|---|---|
| Module | UWB ~500 MHz Bandwidth (BW) pulsed transceiver |
| | chip and impulse response |
| Construction | Head and neck clam shell, shape conformal to human, |
| | saving weight |
| Absorber | Absorber inside chamber with modules embedded |
| Frequency | Operating Frequency band choices across 3.5-9 GHz |
| | with ~500 MHz BW |
| Matching | Much thinner layer thanks to higher frequency and |
| Liquid | with honey comb structure plus perforations for liquid |
| | freedom and a much thinner absorbing layer inside |
| | helmet |
| Visor for | Visor perhaps with entertainment but certainly |
| comfort | breathable and somewhat transparent to room |
| | conditions |
| Image | Transfer function between measurements and images |
| construction | established for all image cases prior to measurement |
| | and looked-up using an AI-large compute once then |
| | small compute for each use |
| Prediction | FDTD commensurate with impulse response |
| | prediction-much richer separation of propagation |
| | paths leading to image resolution and medical |
| | conspicuity |
| Calibration | Delay, gain and phase alignment to remove incidental |
| | electrical component variation and drift |
| Locationing | Full unpowered cellular (coarse) and UWB (fine) |
| | locationing for finding a nearby device anywhere on |
| | earth |

As previously described, the imaging device 10 can comprise a helmet 12, such as a clam shell or crash helmet design, as shown in FIGS. 4A-4C. In some examples, the helmet 12 shape is not just ergonomic, but also supportive of field robustness and intuitive usability. In particular, the clam (e.g., movable portion) of the helmet 12 can be positioned on the front rather than the back for ease of healthcare staff use. In some examples, the helmet 12 can include either an integrated screen or window, which is similar to a microwave oven window, for patient comfort. In some examples, none of the UWB transceivers 24 of the radio frequency array 22 cover the window allowing the patient to clearly see out of the imaging device 10 either directly or through a camera enabled augmented reality. Also, the window can be somewhat visibly transparent, while blocking to RF (to maintain the Faraday cage around the imaging domain) and with a clear or substantially clear matching liquid 20 covering the window.

The helmet 12 can comprise a metalized exterior with easy entry clam shell so that the patient's head can be inserted into the helmet 12 by placing an open end of the back of the clam under the patient's head and closing a lid over the patient's face or part thereof. In some examples, the helmet 12 is configured to feel like a wearable water-bed/pillow surrounding the patient's head, while the membrane 28 of the matching liquid 20 displaces into stretchy pockets around the patient's neck.

As shown in FIGS. 4A-4C, in some examples, the helmet 12 comprises a shell 46 comprising a metal material. The shell 46 can be divided into a front clam-shell portion 48 configured to cover at least a forehead and crown of the patient's head and a rear clam-shell portion 50 pivotally connected to the front clam-shell portion 48 configured to cover an occipital portion of the head. In some examples, as shown in FIG. 4C, the helmet 12 can further comprise a neck extension 52 extending downward from the shell 46 configured to cover a neck, shoulders, and/or clavicle region of the patient for imaging areas of the neck, shoulders, and/or clavicle of the patient.

In some examples, as discussed above, the helmet 12 further comprises a window 54 configured to allow the patient to see objects and/or people outside of the helmet 12. The window 54 can comprise an opening in a shell 46 of the helmet 12 covered by a transparent or translucent panel 56. The panel 56 can comprise and/or be covered by a material, such as a coating or adhered layer, configured to block radio frequency signals maintaining the Faraday cage around the patient's head (e.g., the imaging domain) and ensuring that no radio frequency signals emitted by the UWB transceivers 24 are lost through the panel 56. The radio frequency array 22 can be configured so that none of the UWB transceivers 24 of the array 22 cover the window 54 or translucent panel 56 reducing a likelihood that emitted radio frequency signals would pass through the window 54.

In some examples, the matching liquid 20 can be a clear or substantially clear liquid material enclosed within a transparent or translucent membrane 28. In such instances, the matching liquid 20 and membrane 28 can cover or pass over the interior surface of the panel 56 to accommodate patient heads and faces of different sizes. Including matching liquid 20 covering the window 54 or panel 56 ensures that there are no air gaps between the window 54 and the patient, which could reduce quality of measurement data obtained by the imaging device 10 and/or medical images generated by the imaging device 10.

Alternatively, or in addition to the window 54, the helmet 12 can comprise a visor 58 (shown in FIG. 3) which can be positioned within the field of view of the patient while wearing the imaging device 10. For example, the visor 58 can be pivotally connected to the helmet 12 allowing the patient to move the visor 58 within his or her field of view and, for example, covering the window 54. The visor 58 can comprise a visual display screen 60 configured to display, for example, entertainment content to the patient during an imaging procedure.

As shown in FIGS. 4A and 4B, the helmet 12 can further comprise features for improving patient comfort while wearing the helmet 12. For example, the helmet 12 can comprise one or more air channels 62 (shown in FIG. 4A) extending through the shell 46 for providing external air into the interior cavity 16 defined by the shell 46. The air channels 62 can be configured to provide breathable air to the patient during an imaging procedure, as well as to provide cooling and ventilation to improve comfort. Desirably, the air channels 62 provide sufficient airflow so that the patient can wear the imaging device 10 for extended periods of time (e.g., from several minutes to hour(s)) without discomfort. Also, the air channels 62 may assist in dissipating heat from the interior cavity 16 of the shell 46 further increasing comfort of the patient during an imaging procedure. In some examples, the helmet 12 can further comprise cushions or barriers separating the patient's head from the radio frequency absorbing material 18 and/or membrane 28 containing the matching liquid 20. In some examples, the cushions can surround a perimeter of the window 54 and, as shown in FIG. 4A, can include the air channels 62 allowing air to enter the interior cavity 16 of the shell 46.

FIGS. 5A-6B show another example of the medical imaging device 10 including the radio frequency array 22 of UWB transceivers 24 for medical imaging. Unlike in previous examples including the housing or helmet, the UWB transceivers 24 in FIGS. 5A-6B are enclosed or embedded in a blanket 64, 66 which can be sized to cover a torso, abdomen, and/or extremities (e.g., arms and/or legs) of the patient. Specifically, FIGS. 5A-5C show an upper body blanket 64 covering a patient's torso, abdomen, and arms. FIGS. 6A and 6B show a whole body blanket 66 covering a patient's torso, abdomen, arms, legs, and feet. These examples of the imaging device 10 can be used for obtaining whole body images, as well as images of specific areas of the torso, abdomen, arms, or legs. Whole body imaging may be used for oncology screening, musculoskeletal etc.

In some examples, the blankets 64, 66 may also be configured to wrap around limbs and/or a whole body of the patient meaning, for example, that the blanket 64, 66 can be positioned both above and below a body structure of the patient. For example, the blanket 64, 66 can be structured as a sleeping bag with an opening for the patient to enter and exit the bag. In some examples, the blanket 64, 66 or bag can include zippers, clasps, fasteners, or clips for securing the blanket 64, 66 or bag in place about the patient, which can be unzipped or disconnected to permit easy entry and exit.

In other examples, the blanket 64, 66 can be a component of an imaging assembly including, for example, a blanket 64, 66 configured to be positioned over or above the patient and an absorbing sheet, layer, membrane, or material configured to be positioned underneath the patient. The absorbing sheet can comprise a conductive layer that either includes transceivers for obtaining imaging signals or is configured to reflect signals emitted by UWB transceivers 24 in the blanket 64, 66 towards other UWB transceivers 24 of the blanket 64, 66.

In some examples, the blanket 64, 66 and absorbing sheet can be connected (e.g., stitched together or connected with an adhesive) forming a single imaging device 10. In other examples, the blanket 64, 66 and absorbent sheet can be separate items provided, for example, as components of a medical imaging kit.

As shown in FIGS. 5A-5C, the upper body blanket 64 is a generally rectangular device having a length L1 of about 24 inches to about 48 inches and a width W1 of about 18 inches to 36 inches. As shown in the cross-sectional view of FIG. 5C, the blanket 64 has a similar construction to the helmet 12 and housing 14, as described above. Specifically, the blanket 64 comprises an outer protective layer or backing 68 formed, for example, from a fabric material or flexible plastic sheet. The blanket 64 also includes the radio frequency absorbing material 18 or layer disposed on an inner surface of the backing 68. As previously described, the UWB transceivers 24 of the radio frequency array 22 can be disposed in the radio frequency absorbing material 18 or layer. The blanket 64 can further comprise a layer formed by the matching liquid 20 enclosed within a membrane 28 and disposed on an inner surface of the radio frequency absorbing material 18 or layer. As in previous examples, imaging device 10 can further comprise a celled support structure 30 comprising cells formed from rubberized walls at least partially filled by the matching liquid 20. As in previous examples, the matching liquid 20 or layer is configured to eliminate air gaps between the radio frequency array 22 and the patient ensure that RF signals pass from the UWB transceivers 24 to patient tissue without interference. In some examples, as previously described, the matching liquid 20 or layer and/or the celled support structure 30 can be flexible and compressible so that the matching liquid 20 or layer can accommodate patients of different sizes and body types.

As shown in FIG. 5C, the blanket 64 can further comprise an electronics housing 70, which can be electrically connected to the UWB transceivers 24 of the radio frequency array 22 by wires or via a wireless connection. The electronics housing 70 can contain any or all of the electronic circuitry described above for the imaging device 10. For example, the electronics housing 70 can contain a controller 26 for receiving and processing information about radio frequency signals detected by the UWB transceivers 24 of the radio frequency array 22. The electronics housing 70 can also contain a battery 40 for providing power for the radio frequency array 22 and/or for other electronic components of the imaging device 10. The electronics housing 70 can also contain the communication/location beacon 34 and/or the environmental sensors 42 of the imaging device 10, as previously described.

The imaging device 10 comprising the full length or whole body blanket 66 is shown in FIGS. 6A and 6B. Dimensions of the full length or whole body blanket 66 can be selected to cover all or substantially all of a patient's body from neck to feet. For example, the full length or whole body blanket 66 can have a length L2 of from about 60 inches to about 72 inches and a width of about 18 inches to about 36 inches, though other dimensions could be used for different applications. For example, a whole body blanket 66 configured to wrap around the patient may have a larger width of up to 50 inches or wider.

### Image Processing and Algorithms

Image processing for generating medical images from information detected by the radio frequency array 22 of the imaging device 10 can be performed by the device controller 26 locally on an individual imaging device 10 or by a remote image analysis module or processor disposed, for example, on a remote computing device or computer network. In either case, the image processing algorithms disclosed herein are intended to generate images having millimetric resolution and substantially zero latency based on the received and processed information. In general, the imaging devices 10 of the present disclosure generate medical images by adapting UWB silicon for medical imaging by reversing physics of application of time-of-flight measurement (UWB Silicon), which traditionally use multiple time-of-flight measurements to determine 3D location in free space. By contrast, the imaging devices 10 of the present disclosure are configured to use known 3D locations but unknown materials (e.g. brain tissue) through which propagation occurs to accurately map the unknown materials. While determining location from a small number of time measurements is straightforward, the complexity of 1,000,000 measurements mapping a cube of 20×20×20 cm to 1 mm voxels, as provided by the imaging devices 10 disclosed herein, is orders of magnitude more complex. This is particularly the case because each voxel relates to every other via Maxwell's equations, where tracing rays (CT/MRI) is a poor approximation to the curvature of the electromagnetic fields at the new frequencies and bandwidths.

In some examples, the imaging devices 10 and systems 110 of the present disclosure are configured to process detected data (e.g., RF signals detected by the UWB transceivers 24) for near instantaneous conversion from measurement data to medical images. Near instantaneous conversion means that medical images can be reviewed in real time. Data conversion can be achieved by solving for all possible images in the domain using, for example, a supercomputer (e.g., the NVIDIA DGX GH200) and then training an artificial intelligence algorithm or neural-processing engine to perform the conversion in real time. More specifically, this transfer function pre-calculation means using a supercomputer to pre-calculate the relationship between material properties and time/power measurements, facilitating creation of a large look-up table. In particular examples, the transfer function can be an implementation of a convolutional neural network of a type commonly found in AI applications. Once the transfer function is produced, new measurements are swiftly matched to this table to obtain real-time images.

Through pre-calculation of all conceivable images and their relation to impulse responses across antenna pairs, the imaging devices 10 disclosed herein overcome limitations of real-time iterative solutions. Overcoming such limitations provides improved image fidelity and contrast, with AI offering opportunities for efficient data compression, storage management, and diagnostic aid. In some examples, pre-calculation involves solving all possible electromagnetic problems for every relative permittivity (complex) value for each voxel in the imaging cube and relating that to the 1,000,000 measurements or more that can be made by an imaging device 10 with, for example, about 1,000 UWB transceivers.

In some examples, the algorithm for image conversion functions as follows. First, rather than measuring gain and phase between each transmit and receive antenna, which collapses all the rich propagation path information into two values, the use of UWB allows the direct measurement of a multi-valued impulse response, which can be simulated using Finite-Difference Time-Domain (FDTD) to capture all significant electromagnetic paths and accurately simulate the effect of refractive/reflective interfaces within the imaging domain. In other examples, finite element analysis can also be used for analyzing a set of significant electromagnetic paths between transmitters and receivers of an RF array.

The complex relative permittivity for everything brain-like in the imaging domain goes from around 10 to 90 in the real part and 10 to 50 in the imaginary. Also, significant changes in either of those values are on the order of four meaning that any given voxel can be modelled with about 20 real and about 10 to 20 imaginary quantized values, resulting in about 500 possible values for any given voxel in the imaging domain. The domain itself might be 15×20×25 cm, so there could be a total of 7.5 M voxels that could adopt those values. Each prediction of the domain for each of these cases takes approximately 10 seconds on a ˜100 node cluster of A100s, but the NVIDIA DGX has roughly 3× this performance. In particular, an NVIDIA supercomputer includes 1024 superchips meaning that it can execute around 3,000 of these problems every 10 seconds. The total number of problems is then the 7.5 M voxels multiplied by the number of possible values of the voxel relative permittivity (500). So 3.75e9 and dividing this by 3,000 reveals the number of 10 second blocks needed, which is roughly 1.25 M. So total execution time is about 12.5 M seconds to reach a complete rendition of all possible images converted to the measurements of impulse response one might expect to be yielded by those relative permittivity distributions. Thus, in some instances, roughly half a year of processing is executed in background to acquire a training dataset for an AI. Since an AI can be trained in parallel with the processing, a sparsely populated input matrix may be used to explore an imagining domain space quickly. Following the initial analysis, the processing time can be reduced, due to simplicity of reality relative to the models, to perhaps 1/10th to 1/100th of the time. The trained model can then be transferred to a simpler AI neural engine, such as present on smartphones and laptop computers, and those engines can immediately translate measured impulse responses into an image that can be generated based on measurements collected by the imaging device 10. The calculation may also be executed by neural engines in the cloud.

The processing algorithms performed by the medical imaging device 10 can also include device calibration processes. The purpose of calibration is to ensure accuracy of image and repeatability when fielded despite changes in the state of the electronics used to make measurements, which are incidental with field conditions, such as ambient temperature, etc. In some examples, auto-calibration can be achieved in which effects within the electronics and up to the skull of the patient are subtracted from the measurements made each time the imaging device 10 is used. In examples, since some of the key propagation paths are constant irrespective of the presence of the patient's head, such propagation paths can be used to calibrate out the incidental changes in the delay and gain within the electronics for each use of the imaging device 10. In addition, some unwanted impulse response paths are short and can be excised, while the wanted, optimal for calibration, paths can be used to calibrate all others.

In some examples, calibration can be performed on a per-scan basis. Specifically, since all module-to-module impulse responses are measured, the ones shown as optimal calibration paths can be used to determine precise relative delays within transmitters and receivers, which can then be subtracted out of the paths going through the patient's brain, effectively rendering every use of the imaging device 10 calibrated by the very dataset captured. This calibration is a subtraction of delay and gain components derived from the optimal paths for all other paths used in image reconstruction. The optimal paths are not useful for image reconstruction, because they describe what happens within the matching material, which is constant and known and thus not part of the desired image.

The medical algorithm can also include template based machine learning. For example, prior to availability of massive patient data, there is nonetheless a route for recognizing and highlighting tissues by type or anomaly, e.g. left/right difference between hemispheres can identify candidate stroke regions. Furthermore, thresholding to material properties can refine to blood or blood depleted regions. Such processing can be templatized into filter processes, which are applied to image reconstructions to highlight regions meeting criteria of specific relevance to physicians, much like the segmentation they currently do manually, but in this case the conspicuity of relative permittivity enables automation.

In other examples, AI based learning can be implemented. Specifically, once massive patient data is available, it can be used to correlate images to physician segmented images that contain key medical highlights and thereby automate the expertise of the radiologist in determining diagnostic highlights. In some examples, the medical imaging devices 10 disclosed herein can also be integrated into existing cloud based physician networks. Output from a cloud based/local image reconstruction can be fed back to any DICOM viewer and/or phone app delivering views for captured images.

In one implementation, the imaging device 10 can include a data transfer button. Once the device's colored/lit (e.g., data transfer) button is pushed, image data is captured and transferred to the cloud for processing yielding an image to a browser/AR headset. In some examples, an App on a portable computing device (e.g., an iPhone/iPad) can intercept the processing and use the local neural engine to provide images without first transmitting collected information to the cloud. However, a cloud-based server or device can provide a useful fallback.

In some examples, image studies can be sent to a picture archiving and communication system (PACS) system. Images can also go via the cloud to broader collaborative systems, where many physicians are exposed to generated medical images for diagnosis/consultation. Furthermore, integrating image studies back to a patient's electronic health record (EHR) via existing PACS systems is an assumed integration.

Since the imaging device 10 will be able to tell the network where it is at any time, it may also become an asset, which could be delivered by designated employees or temporary (gig) workers (e.g. grubhub, uber eats). In such examples, the imaging device 10 integrated with a cloud network can be used in a consumer model, where patients obtain periodic preventative scans. Thus, the imaging device 10 can be used for periodic brain scans, as well as for emergency and rescue use.

In some examples, the location information can also be used to track the imaging device 10 as it is being delivered to a customer. For example, a template schedule can be loaded to a locationing unit for each journey, defining a usage location and type. The imaging device 10 can be monitored while in transit via geo-reference, via cellular/GPS to locate the imaging device 10. Furthermore, there could be an opportunity at each cellular/Wi-Fi blink to download a new template profile if location or usage information changes.

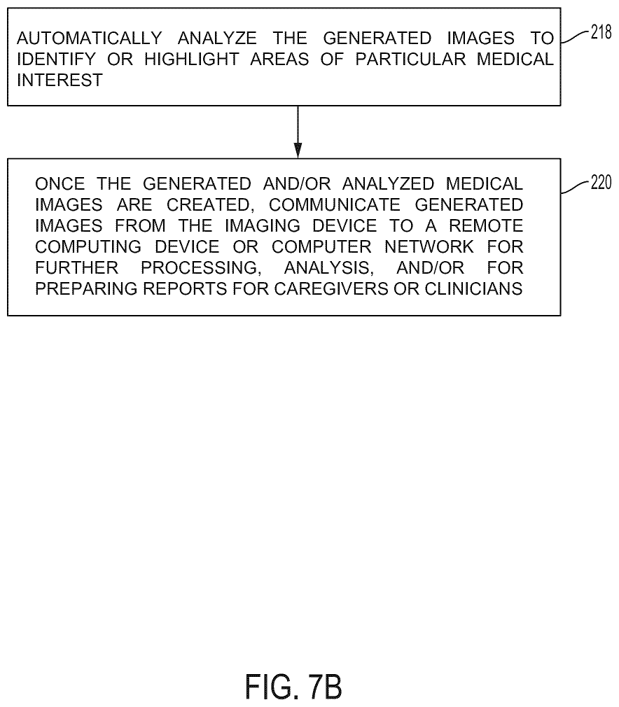

FIGS. 7A and 7B are a flow chart showing steps for processing image data collected by the imaging device 10 of the present disclosure. At step 210, the controller 26 of the imaging device 10 can be configured to cause the UWB transceivers 24 of the radio frequency array 22 to emit pulses of radio frequency signals according to a predetermined impulse response pattern and to detect radio frequency signals traveling along paths between pairs of the UWB transceivers 24 of the radio frequency array 22. In some examples, the impulse response pattern comprises multiple internal reflections, in which some of the multiple internal reflections are damped by absorption, which reduces a number of paths between the pairs of USB transceivers 24 required for the generation of the medical images.

At step 212, the controller 26 can be configured to receive and process information about signal propagation through the interior cavity 16 of the housing 14 from the UWB transceivers 24 of the radio frequency array 22. For example, the received and processed information can comprise information representative of times-of-flight between the pairs of the UWB transceivers 24, which are representative of materials through which propagation of the radio frequency signals has occurred.

At step 214, the controller 26 can be configured to perform an auto-calibration routine based on information about propagation of RF signals through the interior cavity 16 of the housing 14. As previously described, the auto-calibration routine can be provided in order to remove effects within electronics and up to a skull of the patient from detected measurements made by the imaging device 10. In some examples, the auto-calibration can be based on measurements for one or more propagation paths between the pairs of the USB transceivers 24 that occur irrespective of a presence of the patient's head in the interior cavity 16 of the housing 14. In some examples, the one or more propagation paths for calibration can be captured in a data set along with signals for paths between pairs of the UWB transceivers 24 used for generating the medical images. Generally, the one or more propagation paths used for auto-calibration will not be used for generating the medical image.

At step 216, the controller 26 can be configured to generate the medical images based on comparisons between the times-of-flight between the pairs of the UWB transceivers 24 and possible times-of-fight calculated by a model of times-of-flight representative of relative permittivity of tissue. In some examples, generating the medical images can comprise comparing the received and processed information comprising the measured time-of-flight data to a pre-calculated model comprising all conceivable images and their relation to impulse responses across the antenna pairs. As previously described, the pre-calculated model can comprise a model generated by finite difference time domain (FDTD) prediction tools executed across a voxel representation of a 3D space for all possible relative permittivities of the voxel.

In some examples, the pre-calculated model can be applied to measured data by a transfer function. For example, medical images can be generated by applying the transfer function to the received and processed information about the radio frequency signals detected by the UWB transceivers 24 of the radio frequency array 22. The transfer function can be determined by modeling images established for possible image cases detectable by the radio frequency array 22. Also, the transfer function can be selected or modified based on relationships between material properties of tissue within paths between pairs of the plurality of UWB transceivers 24 identified in the modeled images and time/power measurements identified in information detected by the USB transceivers 24. As previously described, the transfer function is based on the image model for the possible images in the imaging domain comprises a model simulated using FDTD to capture all significant electromagnetic paths and simulate effects of refractive/reflective interfaces within the imaging domain.

At step 218, once the medical images are generated, the images can be automatically analyzed to identify or highlight particular areas of medical interest. In order to perform such automated analysis, in some examples, the controller 26 can be configured to apply filter processes to the received and processed information and generate reconstructed medical images based on the filtered information. The reconstructed images can include highlighting or other identifiers to call a reviewer's attention to areas or regions meeting particular criteria and/or that are of particular medical interest. In some examples, highlighting or identification can be based on identified areas having substantial changes in relative permittivity compared to other areas of the generated images. In some examples, regions meeting particular criteria can include areas with right/left differences between hemispheres of the brain, blood or blood depleted regions, and/or candidate stroke regions.

In one example, a filtering process can comprise analyzing the generated images based on previously obtained MRJ/CT to RF comparison data in order to identify areas in the generated medical images corresponding to physician segmented areas identified in a prior MRI/CT dataset. For example, the MRI/CT dataset can include a set of manually analyzed MRI images, in which a physician identifies areas of medical interest. The filtering processes of the present disclosure can be applied to generated RF images that highlight or identify areas in the RF images with an appearance that corresponds to the identified regions of MRI images. In this way, the imaging devices 10 and methods disclosed herein can be adapted for identifying medical conditions or abnormalities previously identified through MRI images, which are more difficult to obtain. In other examples, CT and/or prior electromagnetic tomography data sets can also be used for filtering processes to identify areas of interest in captured images.

At step 220, once the generated and/or analyzed medical images are created, image data can be communicated from the imaging device 10 to a remote computing device or computer network (i.e., the cloud) for further processing, analysis, and/or for preparing reports for caregivers or clinicians. In some instances, data can be uploaded automatically (e.g., immediately after an image is generated) or according to a predetermined communication schedule. In other examples, a user or the patient can initiate a data transmission by, for example, pressing an activation button that causes the wireless data transceiver of the imaging device 10 to initiate transmission of the received and processed information and/or generated images to the remote computing device and/or computer network.

### Cloud-Based Image Analysis System

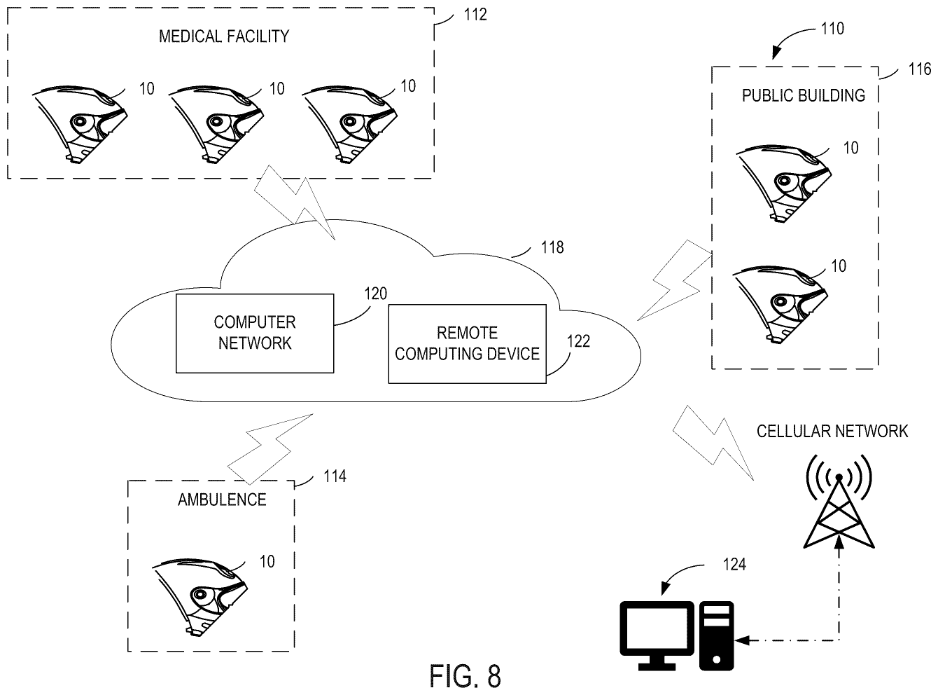

FIG. 8 is a schematic drawing showing a cloud-based image analysis system 110 for multiple imaging devices 10. In some examples, the cloud-based system 110 receives generated images from the multiple imaging devices 10 and distributes the images to caregivers or physicians for interpretation. It is believed that providing cloud remote physician support ensures accurate and timely interpretation. In some examples, the cloud-based image analysis system 110 may also perform some or all of the previously described image processing functions remotely based on measured information received from the multiple imaging devices 10. In some examples, the imaging devices 10 of the present disclosure can be used both in a medical facility and as a portable device for use outside of a hospital system (e.g., for emergency rescue uses). For example, as previously described, the imaging devices 10 can be deployed to public areas for use during emergencies, similar to how AEDs and epi-pens are currently used.

As shown in FIG. 8, the system 110 includes several imaging devices 10 located in a medical facility 112 such as a hospital, long-term care facility, or outpatient treatment facility. Other imaging devices 10 are deployed in an ambulance 114 for use by acute care providers during medical emergencies. Other imaging devices 10 are deployed in public buildings 116 for use by untrained caregivers or bystanders during a medical event.

As previously described, the imaging devices 10 comprise wireless communication circuitry, such as a cellular transmitter 36 or another long-range data transceiver (e.g., Wi-Fi), for transmitting measurement information and/or generated medical images to remote devices. In particular, the imaging devices 10 can be configured to transmit the measurements or generated images to a remote source (e.g., the cloud 118), such as a computer network 120 or remote computing device 122. In some examples, image generation, processing, and/or analysis can be performed by image processing circuitry located on individual imaging devices 10, as described previously. In that case, generated images and/or annotated images can be transmitted to the computer network 120 so that they can be accessed or downloaded by caregivers, physicians, and other interested parties.

In other examples, some or all of the previously described image processing actions can be performed over the computer network 120 and/or by the remote computing device 122. For example, the imaging devices 10 can be configured to measure or detect information representative of times-of-flight between pairs of the UWB transceivers 24 of the radio frequency array 22, which are representative of materials through which propagation of the radio frequency signals has occurred. The computer network 120 or remote computing device 122 can be configured to receive the measured or detected information and analyze the information to generate medical images. For example, the computer network 120 and/or remote computing device 122 can be configured to generate medical images based on comparisons between the times-of-flight between the pairs of the UWB transceivers 24 and possible times-of-fight calculated by a simulated model of possible times-of-flight representative of relative permittivity of tissue, as previously described. The computer network 120 and/or remote computing device 122 can also be configured to analyze and automatically annotate captured images to, for example, identify (e.g., highlight) areas of medical interest in captured images. For example, the computer network 120 and/or remote computing device 122 can be configured to apply filter processes to the received and processed information and generate reconstructed medical images based on the filtered information in which areas or regions meeting particular criteria are highlighted or otherwise identified. In particular, as previously described, image processing for generated medical images can include highlighting or identifying areas of generated medical images with substantial changes in relative permittivity compared to other areas of the generated images. Areas of generated images with right/left differences between hemispheres, brain, blood or blood depleted regions, and/or candidate stroke regions can also be identified.

The system 110 can further comprise a report or feedback device 124 in communication with the computer network 120 and/or remote computing device 122 of the cloud 118 for viewing medical images generated by the computer network 120 or remote computing device 122. The feedback device 124 can be, for example, a smart phone, computer tablet, laptop, computer, or similar computing device comprising a visual display for displaying the generated images. The feedback device 124 can be configured to receive images and other information from the computer network 120 and/or remote computing device 122. The feedback device 124 can be operated by a physician or another caregiver, allowing the caregiver to review the generated medical images to aid in diagnosis and/or patient treatment. In some examples, the feedback device 124 can be configured to execute a software application (e.g., an App) for downloading the generated images from the computer network 120 and causing the images to be displayed on the visual display of the feedback device 124. The App can also be configured to display other patient information, such as patient physiological information or vital signs, along with medical images captured by the imaging device. In other examples, the feedback device 124 can be an augmented reality device, such as Microsoft Hololens® device, configured to project generated images within a field of view of a physician or caregiver wearing the augmented reality device. The physician or caregiver can manipulate the generated images by, for example, repositioning, zooming in or out, or changing contrast or color of generated images projected by the augmented reality device in order to assist in interpretation of the displayed images.

As previously described, the imaging devices 10 can also include the communication/location circuitry or beacons 34 for transmitting location information from the imaging devices 10 to the remote source, such as the computer network 120 and/or remote computing device 122 on the cloud 118. Such location information can be reviewed to ensure that imaging devices 10 are deployed at correct locations and/or to track delivery status of imaging devices being moved to different locations. In some examples, as previously described, location information can be transmitted by cellular and/or via a UWB gateway. In other examples, location information can be transmitted by E-911, Wi-Fi sniff, or over other wireless communication networks, as are known in the art.

### Treatment Methods Using Portable Medical Imaging Devices

The medical imaging devices 10 of the present disclosure can be used for obtaining medical images for diagnosis and treatment of various medical conditions. As discussed above, benefits of the portable medical imaging devices 10 of the present disclosure include improved accessibility, greater safety, and reduced costs. In particular, as discussed above, the imaging devices 10 disclosed herein can be deployed in many locations meaning that medical imaging can be more widely and conveniently available for patients at different locations. Also, images for particular patients can be obtained more frequently to better monitor changes in patient condition. In emergency situations, medical images can be obtained prior to arrival at a medical facility so that patient treatment can be determined and implemented more quickly than occurs currently. Several examples of patient treatment protocols or methods using the medical imaging devices 10 of the present disclosure will now be described showing potential beneficial implementations for the imaging devices 10 of the present disclosure.

In some examples, images generated by the imaging devices 10 disclosed herein can be used for identifying Alzheimer's affected tissue well in advance of symptoms. For example, images could be periodically captured for a population of patients (e.g., all patients over 50 years old) to identify at risk patients. The captured images can be manually or automatically interpreted or analyzed to determine a characteristic of Alzheimer's tissue in the captured images. For example, the characteristic of Alzheimer's tissue can include identifying and/or quantifying a frequency or concentration of amyloid beta plaques. Once the characteristic(s) of Alzheimer's tissue is detected or identified, a treatment method can include providing a medication at a dosage determined based, at least in part, on the analysis of the captured images.

As described in further detail herein, the imaging devices 10 disclosed herein can also be used for monitoring patient condition when an Alzheimer's patient is being treated with drug therapy, such as with an aggressive Amyloid and Tau tangle removing drug, such as lecanemab. For example, a patient identified as being at risk by the screening method described above, but who has not yet started to experience cognitive decline symptom, can begin a drug regimen using lecanemab. During the course of therapy, the patient would normally get 5-12 MRI scans over the year. Scans can also be performed by the imaging devices 10 disclosed herein. The goal of such imaging procedures is to identify ARIA. ARIA is characterized by small clusters of micro-hemorrhages (hemorrhages below 5 mm in diameter), which can be identified through captured medical images with millimetric resolution. If those hemorrhages are seen (as occurs in about 21% of cases) then the drug treatment is titrated until the brain recovers. Once captured images show that the brain has recovered from the detected micro-hemorrhages, the whole process of drug treatment and monitoring with medical images can start again. In some examples, Alzheimer's affected tissue may also be identified while analyzing captured images for AIRA. In some cases, progression of Alzheimer's tissue over time may be monitored to assess effectiveness of the drug therapy.

Images obtained from the medical imaging device 10 of the present disclosure can also be used for blood detection/absence, which is useful for tumor, TBI, and stroke detection and monitoring. In a particular example, the medical imaging devices 10 can be used for early tumor detector. Beneficially, the imaging device 10 may be used for simultaneous detection of head and neck tumors using images simultaneously obtained by the same imaging device 10.

The imaging devices 10 of the present disclosure can also be used for emergency traumatic brain injury diagnosis and treatment and/or stroke triage, such as for determination of ischemic or hemorrhagic condition for stroke treatment. It is believed that the medical imaging devices 10 can change the standard of care for traumatic brain injury. Mobile stroke unit trials were found to result in 25% fewer deaths and halved disability figures simply due to early treatment enabled by on-ambulance imaging. Beneficially, the imaging devices 10 can be small enough to be deployed to ambulances, unlike CT and MRI image devices, which require entire rooms or sizable trucks. Therefore, the imaging devices 10 can be used to triage non-stroke cases and/or to advance stroke cases to immediate treatment during vitals check.

It is also known that post-treatment, undetected complications can result in death following treatment, with decreasing probability as time goes but with still significant risk months to years later. However, this result can be avoided by monitoring the patient, for example, initially every four hours to identify dangerous conditions (e.g., internal bleeding, stroke, loss of blood flow, etc.). Periodically monitoring ischemic patients (e.g., once a day) might also avoid in-patient relapse via alerts to possible hemorrhagic transformation. Due to portability and low cost for obtaining images, the imaging devices 10 can be used for such periodic patient monitoring, which would be difficult to perform using conventional CT/MRI. In some examples, images obtained by the medical imaging device 10 can also be used for easy monthly out-patient scans.

In some examples, a method for triage for a patient who has suffered and/or is suffering a stroke comprises a step of capturing one or more medical images for the patient using the medical imaging device 10 of the present disclosure. The medical images can be obtained, for example, at a scene of an emergency event (i.e., where the stroke occurred). In other examples, images can be obtained in an ambulance during transport to a medical facility, when the patient arrives at an emergency room of the medical facility, or following patent admission to the facility.

Once medical images are captured, the method further comprises analyzing the captured images to, for example, distinguish between ischemic stroke and hemorrhagic stroke. Once the type of stroke is identified, the method can comprise providing appropriate patient treatment for the identified type of stroke and/or monitoring the patient based on the identified type of stroke. For example, monitoring ischemic patients can include capturing new medical images once per day in order to detect instances of relapse or possible hemorrhagic transformation. Monitoring can also include providing periodic alerts to the patient requesting that the patient undergo additional medical imaging. For hemorrhagic patients, monitoring can include capturing new images hourly for the first 24 to 48 hours following stroke in order to identify and/or prevent relapse. Alerts may also be provided to the patient requesting that the patient undergo addition treatment such as, for example, blood pressure monitoring and/or actions for blood pressure control.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements. Furthermore, it is to be understood that the present invention contemplates that, to the extent possible, one or more features of any embodiment can be combined with one or more features of any other embodiment.

The invention claimed is:

1. A portable medical imaging device, comprising:
a housing defining an interior cavity sized to receive a head of a patient;
a radio frequency absorbing material positioned in the interior cavity of the housing;
a radio frequency matching liquid configured to be positioned between the radio frequency absorbing material and the patient's head received within the interior cavity of the housing;
a radio frequency array comprising a plurality of ultra-wide band (UWB) transceivers distributed in the radio frequency absorbing material configured to direct radio frequency signals towards the patient's head and to detect the radio frequency signals emitted by other transceivers of the plurality of UWB transceivers; and
at least one controller configured to receive and process information about the radio frequency signals detected by the plurality of UWB transceivers for generation of medical images of the patient's head.

2. The portable medical imaging device of claim 1, wherein the housing comprises a helmet configured to be used for at least one of the following diagnostic purposes: Alzheimer's screening, amyloid rated imaging abnormalities (ARIA) monitoring, evaluation of traumatic brain injury (TBI), tumor screening, detection of stroke, detection of migraines, or detection of concussions, the helmet comprising a front clam-shell portion configured to cover at least a forehead and crown of the head and a rear clam-shell portion pivotally connected to the front clam-shell portion configured to cover an occipital portion of the head.

3. The portable medical imaging device of claim 2, wherein the helmet comprises a window configured to allow the patient to see objects and/or people outside of the helmet, the window comprising an opening in a shell of the helmet covered by a transparent or translucent pane comprising a material configured to block the radio frequency signals from the plurality of UWB transceivers.

4. The portable medical imaging device of claim 1, wherein the imaging device is configured to be carried by a single staff member of a medical facility.

5. The portable medical imaging device of claim 1, wherein the radio frequency absorbing material comprises doped rubber supporting the plurality of UWB transceivers, and
the matching liquid comprises a liquid material having a permittivity configured to match brain permittivity and positioned to avoid air gaps between the plurality of UWB transceivers and the patient.

6. The portable medical imaging device of claim 1, further comprising a compressible celled support comprising a plurality of cells containing the matching liquid and a liquid reservoir in fluid communication with the plurality of cells configured so that matching liquid passes between the plurality of cells and the reservoir to accommodate heads of different sizes.

7. The portable medical imaging device of claim 1, wherein the radio frequency array comprises at least 300 UWB transceivers, and
wherein the plurality of UWB transceivers operate at a frequency of about 3 GHz to about 8 GHz.

8. The portable medical imaging device of claim 1, wherein the received and processed information comprises information representative of times-of-flight between pairs of the plurality of UWB transceivers, which are representative of materials through which propagation of the radio frequency signals has occurred.

9. The portable medical imaging device of claim 1, wherein the at least one controller is configured to generate the medical images by applying a transfer function to the received and processed information about the radio frequency signals detected by the plurality of UWB transceivers, the transfer function comprising a function determined by modeling images established for all possible image cases detectable by the radio frequency array.

10. The portable medical imaging device of claim 1, wherein the at least one controller is configured to auto-calibrate in order to remove effects within electronics and up to a skull of the patient from detected measurements made by the device, and
wherein auto-calibration is based on measurements for one or more propagation paths between pairs of the plurality of USB transceivers that occur irrespective of a presence of the patient's head in the interior of the housing.

11. The portable medical imaging device of claim 1,
wherein the at least one controller is further configured to apply a filter process to the received and processed information and generate reconstructed medical images based on the filtered information in which areas meeting particular criteria are highlighted or otherwise identified, and
wherein the filtering process by the at least one controller comprises analyzing the generated medical image based on previously obtained MRI/CT to RF comparison data in order to identify areas in the generated medical image corresponding to physician segmented areas identified in a prior MRI/CT dataset.

12. The portable medical imaging device of claim 1, further comprising at least one location beacon configured to transmit radio frequency signals indicating a location of the device, the at least one location beacon comprising:
at least one cellular transmitter for transmission of wireless signals via a cellular network; and
at least one ultra-wide band (UWB) transmitter for transmission of radio signals over a short-range network.

13. The portable medical imaging device of claim 12, further comprising a battery, wherein the at least one controller is configured to apportion battery power from the battery by radio type providing about 90% for the at least one cellular transmitter and less than about 10% to the at least one UWB transmitter.

14. The portable medical imaging device of claim 12, further comprising at least one environmental sensor disposed on the housing configured to detect acceleration and temperature information for the device, and
wherein the at least one controller is configured to modify power usage of the at least one cellular transmitter and/or the at least one UWB transmitter based on the detected acceleration and temperature information.

15. The portable medical imaging device of claim 1, further comprising a wireless data transceiver configured to wirelessly transmit received and processed information collected by the device to a remote computing device and/or computer network.

16. A portable medical imaging device, comprising:
a blanket comprising an outwardly facing surface and an inwardly facing surface configured to be positioned proximate to a patient;
a radio frequency absorbing material positioned in the blanket between the inwardly facing surface and the outwardly facing surface of the blanket;
a radio frequency matching liquid configured to be positioned between the radio frequency absorbing material and the patient, while the patient is covered by the blanket;
a radio frequency array comprising a plurality of ultra-wide band (UWB) transceivers distributed in the radio frequency absorbing material configured to direct radio frequency signals to the patient and to detect the radio frequency signals emitted by other transceivers of the plurality of UWB transceivers; and
at least one controller configured to receive and process information about signals detected by the plurality of UWB transceivers to generate medical images.

17. A tracking and data collection system for medical imaging, the system comprising:
a plurality of the portable medical imaging devices of claim 1; and
a computer server configured to receive and process signals communicated from the plurality of imaging devices, determine a location for one or more of the plurality of portable medical imaging device based on the received and processed signals, generate medical images based on the received and processed signals, and store the generated medical images in electronic medical records associated with one or more of a plurality of patients.

18. A method of treatment for a patient suspected of having and/or diagnosed with Alzheimer's disease, comprising:
capturing at least one medical image of the patient using the portable medical imaging device of claim 1;
analyzing the captured at least one medical image to determine a characteristic of Alzheimer's tissue visible in the captured at least one medical image; and
providing at least one medication at a dosage determined based, at least in part, on the analysis of the captured at least one medical image.

19. The method of claim 19, wherein analysis of the captured at least one medical image identifies Amyloid-Related Imaging Abnormalities (ARIA) in the captured at least one medical image, and
wherein providing the at least one medication comprises administering a drug targeting amyloid beta plaques to the patient.

20. A method of triage for a patient who has suffered and/or is suffering a stroke, comprising:
capturing at least one medical image of a head of the patient using the portable medical imaging device of claim 1;
analyzing the captured at least one medical image to distinguish between areas of the at least one captured medical image evidencing an ischemic stroke and a hemorrhagic stroke; and
treating the patient for either the identified ischemic stroke or the identified hemorrhagic stroke.
